(19) United States
(12) Patent Application Publication
(10) Pub. No.: US 2021/0155994 A1
(43) Pub. Date: May 27, 2021

(54) BIOMARKER FOR DETERMINING AGING, DETERMINING OBESITY AND DIAGNOSING CANCER AND DIAGNOSTIC KIT USING THE SAME

(71) Applicant: KOREA UNIVERSITY RESEARCH AND BUSINESS FOUNDATION, Seoul (KR)

(72) Inventors: Joong-Jean PARK, Seoul (KR); Geon Ho LEE, Seoul (KR); Kee-Ho LEE, Seoul (KR); Eun-Ran PARK, Seoul (KR); Yang Hyun KIM, Seoul (KR)

(73) Assignee: KOREA UNIVERSITY RESEARCH AND BUSINESS FOUNDATION, Seoul (KR)

(21) Appl. No.: 17/118,909

(22) Filed: Dec. 11, 2020

Related U.S. Application Data
(62) Division of application No. 15/746,927, filed on Jan. 24, 2019, now abandoned.

(30) Foreign Application Priority Data
Jul. 24, 2015 (KR) 10-2015-0105253
Jul. 24, 2015 (KR) 10-2015-0105259
Jul. 24, 2015 (KR) 10-2015-0105261

(51) Int. Cl. C12Q 1/6886

(52) U.S. Cl. CPC C12Q 1/6886; C12Q 2600/112

(57) ABSTRACT

The present disclosure relates to a biomarker capable of detecting the genes T3dh (type Ill alcohol dehydrogenase, CG3425), fbp (fructose 1,6-bisphosphatase, CG31692) and AGL (amylo-alpha-1,6-glucosidase, 4-alpha-glucanotransferase, CG9485), which are involved in the induction and occurrence of aging, obesity and cancer, and thereby determining the progression of aging, determining obesity and diagnosing cancer rapidly, accurately and simply. The biomarker may be used to analyze or diagnose the progression of aging, cancer and obesity in a human, a non-human mammal or an insect individually or collectively.

### CROSS-REFERENCE TO RELATED APPLICATIONS

This application is a divisional of U.S. application Ser. No. 15/746,927, having a § 371(c) date of Jan. 24, 2019, which is a § 371 national stage entry of International Application No. PCT/KR2016/008024, filed on Jul. 22, 2016, which claims priority to South Korean Patent Application No. 10-2015-0105259, filed on Jul. 24, 2015, South Korean Patent Application No. 10-2015-0105261, filed on Jul. 24, 2015, and South Korean Patent Application No. 10-2015-0105253, filed on Jul. 24, 2015, the entire contents of which are incorporated herein by reference.

### SEQUENCE LISTING

The instant application contains a Sequence Listing which has been filed electronically in ASCII format and is hereby incorporated by reference in its entirety. Said ASCII copy, created on January 21, 2019, is named G1035-11402_SL.txt and is 142,383 bytes in size.

### TECHNICAL FIELD

The present disclosure relates to a biomarker containing the genes T3dh (type III alcohol dehydrogenase, CG3425), fbp (fructose 1,6-bisphosphatase, CG31692) and AGL (amylo-alpha-1,6-glucosidase, 4-alpha-glucanotransferase, CG9485), which are commonly involved in aging, obesity and cancer, more particularly to a diagnostic kit, etc. using the biomarker.

### BACKGROUND ART

Researches about aging-regulating genes began in early 1990s and are being actively carried out at present. Currently known aging-regulating genes can be classified into reactive oxygen sequestering systems (catalase, superoxide dismutase, etc.), insulin/IGF-1 signaling systems (insulin, InR, PI3K, Akt, Foxo, etc.), gene expression inhibiting systems (sirtuin, etc.), tumor suppressing systems (p53, etc.), material transport systems (sodium dicarboxylate cotransporter, etc.), telomere regulating systems, etc. The genes belonging to these systems are closely involved in the regulation of aging.

With aging, the occurrence of cancer also increases. Interestingly, some tumor suppressor genes such as p53 are also involved in the regulation of aging. The genes known to be associated with carcinogenesis include Ras genes having GTPase activity (Ras, Rac, Rap1, Rata, Rhoa, etc.), Akt-related genes having serine/threonine kinase activity (Akt/PKB, PKC, PKA, RAF, etc.), hedgehog-related genes, protooncogenes such as c-Myc, etc. Also, p53, NFkB, etc. are known as tumor suppressor genes. Especially, HGF and its receptor HGFR (C-Met) are mainly associated with liver cancer. The tumor suppressor gene PTEN inhibits the activity of PI3K. When PTEN is overexpressed, the activity of the insulin/IGF-1 signaling system is decreased and lifespan is often increased. All of these genes were observed from the model organisms of yeast, nematodes, drosophila, mouse, etc. and human genes regulating cancer and aging together have not been researched a lot.

Also, the occurrence of obesity increases with aging. The causes of obesity related with aging include lack of exercise, decreased secretion of growth hormones (GH) and thyroid hormone, etc. Reduced GH secretion leads to decrease in the metabolic effect of GH of degrading carbohydrates, fats, proteins, etc., resulting in decreased muscle mass and accumulation of fats. Because GH increases the secretion of IGF-1 in the liver, the decreased GH secretion due to aging changes the activity of the insulin/IGF-1 signaling system and may be associated with the regulation of lifespan. For example, FIRKO mice with the insulin receptor removed from fat cells do not show accumulation of fats even after overeating and the drosophila aging model shows that body fat increases with aging. Accordingly, obesity and aging are closely related with each other. However, researches on human genes that regulate aging and obesity together are not enough.

That is to say, at present, the efforts to develop a biomarker, a diagnostic kit, a screening method, etc. capable of detecting genes that regulate aging, cancer and obesity collectively and diagnosing aging, cancer and obesity collectively by investigating the expression of these genes are not enough.

As a prior art document related with the present disclosure, Korean Patent Publication No. 10-2012-0021401 (patent document 1) discloses a lifespan-extended transgenic animal and a method for preparing the same by overexpressing the Atg5 gene. However, nothing is disclosed or suggested in the patent document 1 about a biomarker, a diagnostic kit, a screening method, etc. using a gene which regulates aging, cancer and obesity collectively.

### DISCLOSURE

### Technical Problem

The present disclosure is directed to providing a biomarker capable of determining the progression of aging, determining obesity and diagnosing cancer rapidly, accurately and simply.

The present disclosure is also directed to providing a kit and a method for determining or diagnosing using the same.

### Technical Solution

In an aspect, the present disclosure provides a biomarker for determining the progression of aging, containing one or more base sequence selected from a group consisting of base sequences of SEQ ID NOS 1-11, base sequences complementary thereto and mRNAs thereof.

In another aspect, the present disclosure provides a biomarker for determining obesity, containing one or more base sequence selected from a group consisting of base sequences of SEQ ID NOS 1-11, base sequences complementary thereto and mRNAs thereof.

In another aspect, the present disclosure provides a biomarker for diagnosing cancer, containing one or more base sequence selected from a group consisting of base sequences of SEQ ID NOS 1-11, base sequences complementary thereto and mRNAs thereof.

In another aspect, the present disclosure provides a biomarker for determining the progression of aging, determining obesity and diagnosing cancer at the same time, containing one or more base sequence selected from a group consisting of base sequences of SEQ ID NOS 1-11, base sequences complementary thereto and mRNAs thereof.

In another aspect, the present disclosure provides a kit for determining the progression of aging, containing the biomarker; and a hybridization solution.

In the kit for determining the progression of aging, the biomarker is dispersed in a solution or exists in the form of a microarray immobilized on a substrate.

In another aspect, the present disclosure provides a kit for determining obesity, containing the biomarker; and a hybridization solution.

In the kit for determining the progression of aging, the biomarker is dispersed in a solution or exists in the form of a microarray immobilized on a substrate.

In another aspect, the present disclosure provides a kit for diagnosing cancer, containing the biomarker; and a hybridization solution.

In the kit for determining the progression of aging, the biomarker is dispersed in a solution or exists in the form of a microarray immobilized on a substrate.

In another aspect, the present disclosure provides a kit for determining the progression of aging, determining obesity and diagnosing cancer at the same time, containing the biomarker; and a hybridization solution.

In another aspect, the present disclosure provides a method for determining the progression of aging, including: I) a step of isolating and extracting RNA from a diagnosed subject; II) a step of hybridizing the isolated RNA or cDNA synthesized therefrom with a biomarker by contacting the RNA or cDNA with the kit for determining the progression of aging; and III) a step of detecting the degree of hybridization between the biomarker and the RNA or cDNA.

In another aspect, the present disclosure provides a method for determining obesity, including: I) a step of isolating and extracting RNA from a diagnosed subject; II) a step of hybridizing the isolated RNA or cDNA synthesized therefrom with a biomarker by contacting the RNA or cDNA with the kit for determining obesity; and III) a step of detecting the degree of hybridization between the biomarker and the RNA or cDNA.

In another aspect, the present disclosure provides a method for diagnosing cancer, including: I) a step of isolating and extracting RNA from a diagnosed subject; II) a step of hybridizing the isolated RNA or cDNA synthesized therefrom with a biomarker by contacting the RNA or cDNA with the kit for diagnosing cancer; and III) a step of detecting the degree of hybridization between the biomarker and the RNA or cDNA.

In another aspect, the present disclosure provides a method for determining the progression of aging, determining obesity and diagnosing cancer at the same time, including: I) a step of isolating and extracting RNA from a diagnosed subject; II) a step of hybridizing the isolated RNA or cDNA synthesized therefrom with a biomarker by contacting the RNA or cDNA with the kit for determining the progression of aging, determining obesity and diagnosing cancer at the same time; and III) a step of detecting the degree of hybridization between the biomarker and the RNA or cDNA.

### Advantageous Effects

The present disclosure relates to a biomarker capable of detecting the genes T3dh (type III alcohol dehydrogenase, CG3425), fbp (fructose 1,6-bisphosphatase, CG31692) and AGL (amylo-alpha-1,6-glucosidase, 4-alpha-glucanotransferase, CG9485), which are involved in the induction and occurrence of aging, obesity and cancer, and thereby determining the progression of aging, determining obesity and diagnosing cancer rapidly, accurately and simply. The biomarker may be used to analyze or diagnose the progression of aging, cancer and obesity in a human, a non-human mammal or an insect individually or collectively.

### BRIEF DESCRIPTION OF DRAWINGS

FIG. 1 shows the lifespan curve of Actin-GS-Gal4/+W11118 depending on treatment with RU486.

FIG. 2 shows that the quantity of T3dh mRNA is decreased when the expression of T3dh is suppressed using Actin-GS-Gal4 and UAS-T3dh RNAi.

FIG. 3 shows the lifespan curve of Actin-GS-Gal4/+W11118 depending on treatment with RU486.

FIG. 4 shows that the quantity of AGL mRNA is decreased when the expression of AGL is suppressed using Actin-GS-Gal4 and UAS-fbp RNAi.

FIG. 5 shows the lifespan curve of Actin-GS-Gal4/+W11118 depending on treatment with RU486.

FIG. 6 shows that the quantity of AGL mRNA is decreased when the expression of AGL is suppressed using Actin-GS-Gal4 and UAS-AGL RNAi.

FIG. 7 shows that lifespan is curtailed when the expression of T3dh is decreased.

FIG. 8 shows the change in triglyceride content of wild-type drosophila fed with RU486.

FIG. 9 shows the change in triglyceride content of Actin-GS-Gal4/+; UAS-T3dh RNAi/+ drosophila fed with RU486.

FIG. 10 shows a confocal microscopic image of the fat body tissue of Actin-GS-Gal4/UAS-nls.GFP drosophila fed with RU486.

FIG. 11 shows a confocal microscopic image obtained after staining the fat of the fat body tissue of Actin-GS-Gal4/UAS-T3dh RNAi drosophila fed with RU486 with Nile red.

FIG. 12 shows a result of comparing the wing length of tumor growth model drosophila (UAS-PI3K; c765-Gal4).

FIG. 13 shows a result of comparing the wing length of tumor growth model drosophila with the expression of the T3dh gene suppressed (UAS-PI3K/+; c765-Gal4/UAS-T3dh RNAi).

FIG. 14 shows a result of comparing the wing area of tumor growth model drosophila with the expression of the T3dh gene suppressed (UAS-PI3K/+; c765-Gal4/UAS-T3dh RNAi).

FIG. 15 shows that lifespan is curtailed when the expression of fbp is decreased.

FIG. 16 shows the change in triglyceride content of wild-type drosophila fed with RU486.

FIG. 17 shows the change in triglyceride content of Actin-GS-Gal4/+; UAS-fbp RNAi/+ drosophila fed with RU486.

FIG. 18 shows a confocal microscopic image of the fat body tissue of Actin-GS-Gal4/UAS-nls.GFP drosophila fed with RU486.

FIG. 19 shows a confocal microscopic image obtained after staining the fat of the fat body tissue of Actin-GS-Gal4/UAS-fbp RNAi drosophila fed with RU486 with Nile red.

FIG. 20 shows a result of comparing the wing length of tumor proliferation model drosophila (UAS-Ras85D; c765-Gal4).

FIG. 21 shows a result of comparing the wing length of tumor growth model drosophila with the expression of the fbp gene suppressed (UAS-Ras85D/+; c765-Gal4/UAS-fbp RNAi).

FIG. 22 shows a result of comparing the wing area of tumor proliferation model drosophila with the expression of the fbp gene suppressed (UAS-Ras85D/+; c765-Gal4/UAS-fbp RNAi).

FIG. 23 shows that lifespan is curtailed when the expression of AGL is decreased.

FIG. 24 shows the change in triglyceride content of wild-type drosophila fed with RU486.

FIG. 25 shows the change in triglyceride content of Actin-GS-Gal4/+; UAS-AGL RNAi/+ drosophila fed with RU486.

FIG. 26 shows a confocal microscopic image of the fat body tissue of Actin-GS-Gal4/UAS-nls.GFP drosophila fed with RU486.

FIG. 27 shows a confocal microscopic image obtained after staining the fat of the fat body tissue of Actin-GS-Gal4/UAS-AGL RNAi drosophila fed with RU486 with Nile red.

FIG. 28 shows a result of comparing the wing length of tumor growth model drosophila (UAS-PI3K; c765-Gal4) and tumor proliferation model drosophila (UAS-Ras85D; c765-Gal4).

FIG. 29 shows a result of comparing the wing length of tumor growth model drosophila with the expression of the AGL gene suppressed (UAS-PI3K/+; c765-Gal4/UAS-AGL RNAi).

FIG. 30 shows a result of comparing the wing area of tumor growth model drosophila with the expression of the AGL gene suppressed (UAS-PI3K/+; c765-Gal4/UAS-AGL RNAi).

FIG. 31 shows a result of comparing the wing length of tumor proliferation model drosophila with the expression of the AGL gene suppressed (UAS-Ras85D/+; c765-Gal4/UAS-AGL RNAi).

FIG. 32 shows a result of comparing the wing area of tumor proliferation model drosophila with the expression of the AGL gene suppressed (UAS-Ras85D/+; c765-Gal4/UAS-AGL RNAi).

### BEST MODE

Hereinafter, the present disclosure is described in more detail.

The present disclosure is directed to providing a biomarker capable of determining the progression of aging of a human, a non-human mammal or an insect rapidly and simply.

An aspect of the present disclosure relates to a biomarker for determining the progression of aging, containing one or more base sequence selected from a group consisting of base sequences of SEQ ID NOS 1-11, base sequences complementary thereto and mRNAs thereof.

The biomarker for determining the progression of aging according to the present disclosure refers to a biomarker capable of qualitatively determining the progression of aging. The biomarker is capable of determining the progression of aging by comparing the expression level of the biomarker measured in a diagnosed subject with the standard expression level of the biomarker in the same species as the diagnosed subject, based on the fact that lifespan, or the phenotype of aging, is curtailed as the expression of one or more base sequence selected from a group consisting of base sequences of SEQ ID NOS 1-11, base sequences complementary thereto and mRNAs thereof is decreased.

Because a human, non-human mammals and insects are genetically different, they exhibit quite different progression of aging. However, the biomarker for determining the progression of aging containing one or more base sequence selected from a group consisting of base sequences of SEQ ID NOS 1-11, base sequences complementary thereto and mRNAs thereof according to the present disclosure can determine the progression of aging of a diagnosed subject rapidly, accurately and simply for individual species.

The base sequence selected from a group consisting of base sequences of SEQ ID NOS 1-11 is not particularly limited as long as it is one extracted from a mutant drosophila with the expression of a particular gene suppressed by the UAS-GLA4 system. Specifically, SEQ ID NO 1 corresponds to the T3dh (type III alcohol dehydrogenase, CG3425) gene of drosophila, SEQ ID NOS 2-5 correspond to the fbp (fructose 1,6-bisphosphatase, CG31692) gene of drosophila and SEQ ID NOS 6-11 correspond to the AGL (amylo-alpha-1,6-glucosidase, 4-alpha-glucanotransferase, CG9485) of drosophila.

Specifically, in the present disclosure, an inducible gene switch (GS) GAL/UAS expression system is used to evaluate the function of candidate genes using drosophila. GAL4 is a protein originally derived from yeast. When GAL4 is expressed after being introduced into drosophila, it binds to DNA sequence called Upstream Activating Sequence (UAS) in the presence of the drug RU486 (mifepristone), thereby activating the transcription of a specific gene downstream the UAS. In the albescence of RU486, the transcription of the specific gene is deactivated. Based on this, the expression of the T3dh, fbp or AGL gene is controlled by regulating the activity of T3dh RNAi, fbp RNAi or AGL RNAi and their function is identified.

When the expression of the one or more base sequence selected from a group consisting of base sequences of SEQ ID NOS 1-11, base sequences complementary thereto and mRNAs thereof is specifically decreased, the aging of drosophila is accelerated.

Specifically, after exposing mutant drosophila with the expression of a particular gene suppressed by the UAS-GAL4 system to RU486, the effect resulting as the expression of the particular gene is decreased by two times or more was demonstrated through repeated experiments. As a result, it was confirmed that, when the expression of the gene containing one or more base sequence selected from a group consisting of base sequences of SEQ ID NOS 1-11, base sequences complementary thereto and mRNAs thereof is decreased by two times or more, aging proceeds further. Accordingly, the gene may be used as a biomarker for determining the aging of a human, a non-human mammal or an insect.

Moreover, because the one or more base sequence selected from a group consisting of base sequences of SEQ ID NOS 1-11, base sequences complementary thereto and mRNAs thereof is homologous to the human ADHFE1 (alcohol dehydrogenase, iron containing, 1, NP_653251.2 467 aa) gene, the biomarker according to the present disclosure may be used to determine the progression of human aging, in addition to insects.

The one or more base sequence selected from a group consisting of base sequences of SEQ ID NOS 1-11 and base sequences complementary thereto is a cDNA lacking an intron. This cDNA is one prepared as a complementary DNA using mRNA produced from genomic DNA through transcription.

That is to say, because the biomarker may be used to determine the progression of aging of a human, a non-human mammal or an insect, it is expected to be useful in determining the progression of aging of a diagnosed subject.

The present disclosure is directed to providing a biomarker capable of determining the obesity of a human, a non-human mammal or an insect rapidly and simply.

Another aspect of the present disclosure relates to a biomarker for determining obesity, containing one or more base sequence selected from a group consisting of base sequences of SEQ ID NOS 1-11, base sequences complementary thereto and mRNAs thereof.

In the present disclosure, the biomarker for determining obesity refers to a biomarker capable of determining the increase in obesity (specifically, increase in triglyceride content and increase in size and density of lipid droplets in fat body tissue) qualitatively. The biomarker is capable of determining the increase in obesity by comparing the expression level of the biomarker measured in a diagnosed subject with the standard expression level of the biomarker in the same species as the diagnosed subject, based on the fact that body fat content and size and density of lipid droplets in fat body tissue increase as the expression of one or more base sequence selected from a group consisting of base sequences of SEQ ID NOS 1-11, base sequences complementary thereto and mRNAs thereof is decreased.

Because a human, non-human mammals and insects are genetically different, they will also show difference in obesity. However, the biomarker for determining obesity containing one or more base sequence selected from a group consisting of base sequences of SEQ ID NOS 1-11, base sequences complementary thereto and mRNAs thereof according to the present disclosure can determine the obesity of a diagnosed subject rapidly, accurately and simply for individual species.

The base sequence selected from a group consisting of base sequences of SEQ ID NOS 1-11 is not particularly limited as long as it is one extracted from a mutant drosophila with the expression of a particular gene suppressed by the UAS-GAL4 system. Specifically, SEQ ID NO 1 corresponds to the T3dh (type III alcohol dehydrogenase, CG3425) gene of drosophila, SEQ ID NOS 2-5 correspond to the fbp (fructose 1,6-bisphosphatase, CG31692) gene of drosophila and SEQ ID NOS 6-11 correspond to the AGL (amylo-alpha-1,6-glucosidase, 4-alpha-glucanotransferase, CG9485) of drosophila.

Specifically, in the present disclosure, an inducible gene switch (GS) GAL/UAS expression system is used to evaluate the function of candidate genes using drosophila. GAL4 is a protein originally derived from yeast. When GAL4 is expressed after being introduced into drosophila, it binds to DNA sequence called Upstream Activating Sequence (UAS) in the presence of the drug RU486 (mifepristone), thereby activating the transcription of a specific gene downstream the UAS. In the albescence of RU486, the transcription of the specific gene is deactivated. Based on this, the expression of the T3dh, fbp or AGL gene is controlled by regulating the activity of T3dh RNAi, fbp RNAi or AGL RNAi and their function is identified.

The expression of the one or more base sequence selected from a group consisting of base sequences of SEQ ID NOS 1-11, base sequences complementary thereto and mRNAs thereof is specifically decreased as the triglyceride content and the size and density of lipid droplets in the fat body tissue of drosophila increase greatly.

Specifically, after exposing mutant drosophila with the expression of a particular gene suppressed by the UAS-GAL4 system to RU486, the effect resulting as the expression of the particular gene is decreased by two times or more was demonstrated through repeated experiments. As a result, it was confirmed that, when the expression of the gene containing one or more base sequence selected from a group consisting of base sequences of SEQ ID NOS 1-11, base sequences complementary thereto and mRNAs thereof is decreased by two times or more, obesity is increased. Accordingly, the gene may be used as a biomarker for determining the obesity of a human, a non-human mammal or an insect.

Moreover, because the one or more base sequence selected from a group consisting of base sequences of SEQ ID NOS 1-11, base sequences complementary thereto and mRNAs thereof is homologous to the human ADHFE1 (alcohol dehydrogenase, iron containing, 1, NP_653251.2 467 aa) gene, the biomarker according to the present disclosure may be used to determine the obesity of humans, in addition to insects.

The one or more base sequence selected from a group consisting of base sequences of SEQ ID NOS 1-11 and base sequences complementary thereto is a cDNA lacking an intron. This cDNA is one prepared as a complementary DNA using mRNA produced from genomic DNA through transcription.

That is to say, because the biomarker may be used to determine the increase in obesity of a human, a non-human mammal or an insect, it is expected to be useful in determining the increase in obesity of a diagnosed subject.

The present disclosure is directed to providing a biomarker capable of diagnosing the cancer of a human, a non-human mammal or an insect rapidly and simply.

Another aspect of the present disclosure relates to a biomarker for diagnosing cancer, containing one or more base sequence selected from a group consisting of base sequences of SEQ ID NOS 1-11, base sequences complementary thereto and mRNAs thereof.

In the present disclosure, the biomarker for diagnosing the cancer refers to a biomarker capable of determining the proliferation or growth of cancer cells and tissues qualitatively. The biomarker is capable of determining the occurrence or proliferation of cancer by comparing the expression level of the biomarker measured in a diagnosed subject with the standard expression level of the biomarker in the same species as the diagnosed subject, based on the fact that the phenotype of tumor growth model drosophila (or tumor proliferation model drosophila) decrease as the expression of one or more base sequence selected from a group consisting of base sequences of SEQ ID NOS 1-11, base sequences complementary thereto and mRNAs thereof is decreased.

Because a human, non-human mammals and insects are genetically different, they will also show difference in occurrence of cancer. However, the biomarker for diagnosing cancer containing one or more base sequence selected from a group consisting of base sequences of SEQ ID NOS 1-11, base sequences complementary thereto and mRNAs thereof according to the present disclosure can determine the occurrence of cancer of a diagnosed subject rapidly, accurately and simply for individual species.

The base sequence selected from a group consisting of base sequences of SEQ ID NOS 1-11 is not particularly limited as long as it is one extracted from a mutant drosophila with the expression of a particular gene suppressed by the UAS-GAL4 system. Specifically, SEQ ID NO 1 corresponds to the T3dh (type III alcohol dehydrogenase, CG3425) gene of drosophila, SEQ ID NOS 2-5 correspond to the fbp (fructose 1,6-bisphosphatase, CG31692) gene of drosophila and SEQ ID NOS 6-11 correspond to the AGL (amylo-alpha-1,6-glucosidase, 4-alpha-glucanotransferase, CG9485) of drosophila.

Specifically, in the present disclosure, an inducible gene switch (GS) GAL/UAS expression system is used to evaluate the function of candidate genes using drosophila. GAL4 is a protein originally derived from yeast. When GAL4 is expressed after being introduced into drosophila, it binds to DNA sequence called Upstream Activating Sequence (UAS) in the presence of the drug RU486 (mifepristone), thereby activating the transcription of a specific gene downstream the UAS. In the albescence of RU486, the transcription of the specific gene is deactivated. Based on this, the expression of the T3dh, fbp or AGL gene is controlled by regulating the activity of T3dh RNAi, fbp RNAi or AGL RNAi and their function is identified.

The expression of the one or more base sequence selected from a group consisting of base sequences of SEQ ID NOS 1-11, base sequences complementary thereto and mRNAs thereof is specifically decreased as the wing phenotype of tumor growth model drosophila (or tumor proliferation model drosophila) is decreased greatly.

Specifically, after exposing mutant drosophila with the expression of a particular gene suppressed by the UAS-GAL4 system to RU486, the effect resulting as the expression of the particular gene is decreased by two times or more (decrease in wing length and area through suppression of the expression of cancer-related PI3K) was demonstrated through repeated experiments. As a result, it was confirmed that, when the expression of the gene containing one or more base sequence selected from a group consisting of base sequences of SEQ ID NOS 1-11, base sequences complementary thereto and mRNAs thereof is decreased by two times or more, the wing phenotype of tumor growth model drosophila is increased. Accordingly, the gene may be used as a biomarker for diagnosing the occurrence of cancer in a human, a non-human mammal or an insect.

Moreover, because the one or more base sequence selected from a group consisting of base sequences of SEQ ID NOS 1-11, base sequences complementary thereto and mRNAs thereof is homologous to the human ADHFE1 (alcohol dehydrogenase, iron containing, 1, NP_653251.2 467 aa) gene, the biomarker according to the present disclosure may be used to diagnose the occurrence or proliferation of cancer in humans, in addition to insects.

The one or more base sequence selected from a group consisting of base sequences of SEQ ID NOS 1-11 and base sequences complementary thereto is a cDNA lacking an intron. This cDNA is one prepared as a complementary DNA using mRNA produced from genomic DNA through transcription.

That is to say, because the biomarker may be used to determine the occurrence and proliferation of cancer in a human, a non-human mammal or an insect, it is expected to be useful in diagnosing the cancer of a diagnosed subject.

Although the biomarker containing one or more base sequence selected from a group consisting of base sequences of SEQ ID NOS 1-11, base sequences complementary thereto and mRNAs thereof may be used to determine aging, determine obesity and diagnose cancer separately, as described above, it may also be used to determine the progression of aging, determine the increase in obesity and detect the occurrence of cancer at the same time.

As described above, as the expression of the one or more base sequence selected from a group consisting of base sequences of SEQ ID NOS 1-11, base sequences complementary thereto and mRNAs thereof is decreased, mutant drosophila with the expression of a particular gene suppressed by the UAS-GAI4 system experiences further progression of aging, increase in triglycerides and the density and size of lipid droplets in fat body tissue and decreased occurrence of cancer. Therefore, the biomarker is capable of determining the progression of aging, determining the increase in obesity and detecting the occurrence of cancer at the same time by comparing the expression level of the biomarker measured in a diagnosed subject with the standard expression level of the biomarker in the same species as the diagnosed subject.

In addition, in the present disclosure, the biomarker may be, not only the base sequences of SEQ ID NOS 1-11, base sequences complementary thereto or mRNAs thereof, but also one or more protein encoded by the base sequences. The protein may be composed of one or more selected from a group consisting of amino acid sequences 12-22.

The protein consisting of the amino acid sequences 12-22 may also determine and diagnose one or more selected from aging, obesity and cancer based on the increased or decreased quantity of the protein as compared to a normal control group (standard expression level of the same species as the diagnosed subject).

A kit using the protein may be a kit for ELISA (enzyme-linked immunosorbent assay) and may detect an antigen-antibody complex quantitatively by contacting an antibody binding specifically to the protein with a biological sample selected from tissue, cell, urine, blood, serum and plasma of a diagnosed subject.

One or more selected from aging, obesity and cancer of the diagnosed subject may be determined and diagnosed by comparing the detection result with the standard protein expression level of the diagnosed subject.

Analytical techniques for measuring the protein expression level include western blot, ELISA (enzyme-linked immunosorbent assay), RIA (radioimmunoassay), radioimmunodiffusion, Ouchterlony immunodiffusion, rocket immunoelectrophoresis, histoimmunostaining, immunoprecipitation, complement fixation assay, FACS, protein chip, etc., although not being limited thereto.

Through these analytical techniques, the amount of the antigen-antibody complex formed in the diagnosed subject can be compared with the standard amount of the antigen-antibody complex formed in the same species as the diagnosed subject and one or more of the progression of aging, the increase of obesity and the occurrence of cancer may be determined or diagnosed by investigating whether the expression level of one or more protein selected from the amino acid sequences 12-22 is increased significantly.

Another aspect of the present disclosure relates to a kit for determining the progression of aging, containing the biomarker and a hybridization solution.

The biomarker is the same as described above and may be dispersed in a solution or immobilized on a substrate with high density. In other words, the biomarker may be in the form of a microarray immobilized on specific regions. The microarray is well known in the art.

By hybridizing the biomarker which is dispersed in the solution or immobilized on the substrate with mRNA or cDNA extracted from a diagnosed subject, the kit may determine whether mRNA or cDNA of the same sequence is expressed.

Therefore, the biomarker used in the kit requires a process wherein one strand of the base sequence is detached.

When the biomarker is in the form of a microarray immobilized on a substrate, the substrate may be any substrate to which a biomarker can be coupled under a condition where the background level of hybridization is maintained low. Usually, the substrate may be a microtiter plate, a membrane (e.g., nylon or nitrocellulose), a microsphere (bead) or a chip.

Before being applied or immobilized onto a membrane, the biomarker may be modified to improve hybridization efficiency. The modification may include homopolymer tailing, coupling with various reactive functional groups such as an aliphatic group, a NH₂ group, an SH group and a carboxyl group or coupling with a biotin, a hapten ora protein.

The “hybridization” refers to a process in which two complementary strands of nucleic acid are combined to form a double-stranded molecule (hybrid).

The hybridization solution is a buffer solution which allows for hybridization of the biomarker with mRNA or cDNA extracted from a diagnosed subject and a solution known in the art may be used.

The kit may further contain a detector capable of detecting the nucleic acid of the diagnosed subject formed from hybridization with the biomarker. As the detector, a scanner, a spectrophotometer, a liquid scintillation counter, etc. may be used, although not being limited thereto. The kit of the present disclosure may further contain an instruction describing the optimal reaction condition.

The kit may qualitatively detecting the progression of aging by comparing the expression level of the biomarker measured for the diagnosed subject with the standard expression level of the biomarker for the same species as the diagnosed subject. Specifically, when the expression level of the biomarker measured for the diagnosed subject is compared with the standard expression level of the biomarker for the same species as the diagnosed subject using the kit, if the expression level is decreased for the diagnosed subject, it may be determined accurately and rapidly that the aging of the diagnosed subject has progressed further as compared to the average aging of the same species.

Another aspect of the present disclosure relates to a kit for determining obesity, containing the biomarker and a hybridization solution.

The biomarker is the same as described above and may be dispersed in a solution or immobilized on a substrate with high density. In other words, the biomarker may be in the form of a microarray immobilized on specific regions. The microarray is well known in the art.

By hybridizing the biomarker which is dispersed in the solution or immobilized on the substrate with mRNA or cDNA extracted from a diagnosed subject, the kit may determine whether mRNA or cDNA of the same sequence is expressed.

Therefore, the biomarker used in the kit requires a process wherein one strand of the base sequence is detached.

When the biomarker is in the form of a microarray immobilized on a substrate, the substrate may be any substrate to which a biomarker can be coupled under a condition where the background level of hybridization is maintained low. Usually, the substrate may be a microtiter plate, a membrane (e.g., nylon or nitrocellulose), a microsphere (bead) or a chip.

Before being applied or immobilized onto a membrane, the biomarker may be modified to improve hybridization efficiency. The modification may include homopolymer tailing, coupling with various reactive functional groups such as an aliphatic group, a NH₂ group, an SH group and a carboxyl group or coupling with a biotin, a hapten ora protein.

The “hybridization” refers to a process in which two complementary strands of nucleic acid are combined to form a double-stranded molecule (hybrid).

The hybridization solution is a buffer solution which allows for hybridization of the biomarker with mRNA or cDNA extracted from a diagnosed subject and a solution known in the art may be used.

The kit may further contain a detector capable of detecting the nucleic acid of the diagnosed subject formed from hybridization with the biomarker. As the detector, a scanner, a spectrophotometer, a liquid scintillation counter, etc. may be used, although not being limited thereto. The kit of the present disclosure may further contain an instruction describing the optimal reaction condition.

The kit may qualitatively detecting obesity by comparing the expression level of the biomarker measured for the diagnosed subject with the standard expression level of the biomarker for the same species as the diagnosed subject. Specifically, when the expression level of the biomarker measured for the diagnosed subject is compared with the standard expression level of the biomarker for the same species as the diagnosed subject using the kit, if the expression level is decreased for the diagnosed subject, it may be determined accurately and rapidly that the average triglyceride content and size and density of lipid droplets of the diagnosed subject are increased as compared to those of the same species.

Another aspect of the present disclosure relates to a kit for diagnosing cancer, containing the biomarker and a hybridization solution.

The biomarker is the same as described above and may be dispersed in a solution or immobilized on a substrate with high density. In other words, the biomarker may be in the form of a microarray immobilized on specific regions. The microarray is well known in the art.

By hybridizing the biomarker which is dispersed in the solution or immobilized on the substrate with mRNA or cDNA extracted from a diagnosed subject, the kit may determine whether mRNA or cDNA of the same sequence is expressed.

Therefore, the biomarker used in the kit requires a process wherein one strand of the base sequence is detached.

When the biomarker is in the form of a microarray immobilized on a substrate, the substrate may be any substrate to which a biomarker can be coupled under a condition where the background level of hybridization is maintained low. Usually, the substrate may be a microtiter plate, a membrane (e.g., nylon or nitrocellulose), a microsphere (bead) or a chip.

Before being applied or immobilized onto a membrane, the biomarker may be modified to improve hybridization efficiency. The modification may include homopolymer tailing, coupling with various reactive functional groups such as an aliphatic group, a NH₂ group, an SH group and a carboxyl group or coupling with a biotin, a hapten ora protein.

The “hybridization” refers to a process in which two complementary strands of nucleic acid are combined to form a double-stranded molecule (hybrid).

The hybridization solution is a buffer solution which allows for hybridization of the biomarker with mRNA or cDNA extracted from a diagnosed subject and a solution known in the art may be used.

The kit may further contain a detector capable of detecting the nucleic acid of the diagnosed subject formed from hybridization with the biomarker. As the detector, a scanner, a spectrophotometer, a liquid scintillation counter, etc. may be used, although not being limited thereto. The kit of the present disclosure may further contain an instruction describing the optimal reaction condition.

The kit may qualitatively diagnosing cancer by comparing the expression level of the biomarker measured for the diagnosed subject with the standard expression level of the biomarker for the same species as the diagnosed subject. Specifically, when the expression level of the biomarker measured for the diagnosed subject is compared with the standard expression level of the biomarker for the same species as the diagnosed subject using the kit, if the expression level is decreased for the diagnosed subject, it may be determined accurately and rapidly that cancer has occurred or grown in the diagnosed subject as compared to the same species.

Although the kit according to the present disclosure may be used to determine aging, determine obesity and diagnose cancer separately, as described above, it may also be used to determine the progression of aging, determine the increase in obesity and detect the occurrence of cancer at the same time.

As described above, the kit contains the biomarker containing one or more base sequence selected from a group consisting of base sequences of SEQ ID NOS 1-11, base sequences complementary thereto and mRNAs thereof and the hybridization solution. As the expression of the biomarker is decreased, mutant drosophila with the expression of a particular gene suppressed by the UAS-GAI4 system experiences further progression of aging, increase in triglycerides and the density and size of lipid droplets in fat body tissue and decreased occurrence of cancer. Therefore, the kit is capable of determining the progression of aging, determining the increase in obesity and detecting the occurrence of cancer at the same time by comparing the expression level of the biomarker measured in a diagnosed subject with the standard expression level of the biomarker in the same species as the diagnosed subject.

Another aspect of the present disclosure relates to a method for determining the progression of aging, including:

I) a step of isolating and extracting RNA from a diagnosed subject;

II) a step of hybridizing the isolated RNA or cDNA synthesized therefrom with a biomarker by contacting the RNA or cDNA with the kit for determining the progression of aging; and

III) a step of detecting the degree of hybridization between the biomarker and the RNA or cDNA.

The isolation of RNA from the diagnosed subject may be performed by a method well known in the art. Specifically, a cell may be isolated from the diagnosed subject and then the RNA may be isolated from the isolated cell of the diagnosed subject in vitro.

In an exemplary embodiment of the present disclosure, the cDNA may be a first strand cDNA synthesized using the isolated RNA as a template. The first strand cDNA may be synthesized by a method commonly employed in the art. For example, it may be synthesized using a reverse transcriptase, an RNase block ribonuclease inhibitor, etc. Examples of the reverse transcriptase include reverse transcriptases derived from various sources, e.g., avian myeloblastosis virus-derived virus reverse transcriptase (AMV RTase), murine leukemia virus-derived virus reverse transcriptase (MMLV RTase) and Rous-associated virus 2 reverse transcriptase (RAV-2 RTase).

Specifically, the cDNA may be labeled with a detectable label. The label may be a material emitting fluorescence, phosphorescence or radiation, although not being limited thereto. Specifically, the label is Cy5 or Cy3. When synthesizing the first strand cDNA, if the synthesis is performed by labeling Cy5 or Cy3 at the 5′-terminal of a primer, a target sequence may be labeled with a detectable fluorescent label. Labeling with a radioactive material may be achieved as follows. When synthesizing the first strand cDNA, if a radioactive isotope such as ³²P, ³⁵S, etc. is added to a reaction solution, the resulting synthesis product may be labeled with the radioactive material.

The step of detecting the degree of hybridization may be performed through capillary electrophoresis, gel electrophoresis, radiation measurement, fluorescence measurement or phosphorescence measurement.

In another exemplary embodiment of the present disclosure, the method for determining the progression of aging may further include a step of determining the progression of aging of the diagnosed subject by comparing the detection result with the standard of the corresponding diagnosed subject.

Meanwhile, the method for determining the progression of aging may be for providing information necessary for determining the progression of aging of the diagnosed subject. By isolating a cell from the diagnosed subject and isolating RNA from the isolated cell of the diagnosed subject in vitro and adding thereto a probe capable of detecting the expression of a base sequence selected from a group consisting of SEQ ID NOS 1-11 or an antibody capable of detecting the expression of an amino acid sequence selected from a group consisting of SEQ ID NOS 12-22, a gene containing a base sequence selected from a group consisting of SEQ ID NOS 1-11 or a protein containing an amino acid sequence selected from a group consisting of SEQ ID NOS 12-22 may be detected.

The method may further include a step of determining the progression of aging of the diagnosed subject by comparing the expression level of the detected gene and protein with the standard of the corresponding diagnosed subject.

The diagnosed subject may be a human, a non-human mammal or an insect.

Another aspect of the present disclosure relates to a method for determining obesity, including:

I) a step of isolating and extracting RNA from a diagnosed subject;

II) a step of hybridizing the isolated RNA or cDNA synthesized therefrom with a biomarker by contacting the RNA or cDNA with the kit for determining obesity; and

III) a step of detecting the degree of hybridization between the biomarker and the RNA or cDNA.

The isolation of RNA from the diagnosed subject may be performed by a method well known in the art. Specifically, a cell may be isolated from the diagnosed subject and then the RNA may be isolated from the isolated cell of the diagnosed subject in vitro.

In an exemplary embodiment of the present disclosure, the cDNA may be a first strand cDNA synthesized using the isolated RNA as a template. The first strand cDNA may be synthesized by a method commonly employed in the art. For example, it may be synthesized using a reverse transcriptase, an RNase block ribonuclease inhibitor, etc. Examples of the reverse transcriptase include reverse transcriptases derived from various sources, e.g., avian myeloblastosis virus-derived virus reverse transcriptase (AMV RTase), murine leukemia virus-derived virus reverse transcriptase (MMLV RTase) and Rous-associated virus 2 reverse transcriptase (RAV-2 RTase).

Specifically, the cDNA may be labeled with a detectable label. The label may be a material emitting fluorescence, phosphorescence or radiation, although not being limited thereto. Specifically, the label is Cy5 or Cy3. When synthesizing the first strand cDNA, if the synthesis is performed by labeling Cy5 or Cy3 at the 5′-terminal of a primer, a target sequence may be labeled with a detectable fluorescent label. Labeling with a radioactive material may be achieved as follows. When synthesizing the first strand cDNA, if a radioactive isotope such as ³²P, ³⁵S, etc. is added to a reaction solution, the resulting synthesis product may be labeled with the radioactive material.

The step of detecting the degree of hybridization may be performed through capillary electrophoresis, gel electrophoresis, radiation measurement, fluorescence measurement or phosphorescence measurement.

In another exemplary embodiment of the present disclosure, the method for determining obesity may further include a step of determining obesity by comparing the increase in triglyceride content and increase in the size and obesity of lipid droplets in fat body tissue with the standard of the corresponding diagnosed subject.

Meanwhile, the method for determining obesity may be for providing information necessary for determining obesity of the diagnosed subject. By isolating a cell from the diagnosed subject and isolating RNA from the isolated cell of the diagnosed subject in vitro and adding thereto a probe capable of detecting the expression of a base sequence selected from a group consisting of SEQ ID NOS 1-11 or an antibody capable of detecting the expression of an amino acid sequence selected from a group consisting of SEQ ID NOS 12-22, a gene containing a base sequence selected from a group consisting of SEQ ID NOS 1-11 or a protein containing an amino acid sequence selected from a group consisting of SEQ ID NOS 12-22 may be detected.

The method may further include a step of determining the increase in obesity (increase in triglyceride content and increase in the size and density of lipid droplets in fat body tissue) of the diagnosed subject by comparing the expression level of the detected gene and protein with the standard of the corresponding diagnosed subject.

The diagnosed subject may be a human, a non-human mammal or an insect.

Another aspect of the present disclosure relates to a method for diagnosing cancer, including:

I) a step of isolating and extracting RNA from a diagnosed subject;

II) a step of hybridizing the isolated RNA or cDNA synthesized therefrom with a biomarker by contacting the RNA or cDNA with the kit for diagnosing cancer; and

III) a step of detecting the degree of hybridization between the biomarker and the RNA or cDNA.

The isolation of RNA from the diagnosed subject may be performed by a method well known in the art. Specifically, a cell may be isolated from the diagnosed subject and then the RNA may be isolated from the isolated cell of the diagnosed subject in vitro.

In an exemplary embodiment of the present disclosure, the cDNA may be a first strand cDNA synthesized using the isolated RNA as a template. The first strand cDNA may be synthesized by a method commonly employed in the art. For example, it may be synthesized using a reverse transcriptase, an RNase block ribonuclease inhibitor, etc. Examples of the reverse transcriptase include reverse transcriptases derived from various sources, e.g., avian myeloblastosis virus-derived virus reverse transcriptase (AMV RTase), murine leukemia virus-derived virus reverse transcriptase (MMLV RTase) and Rous-associated virus 2 reverse transcriptase (RAV-2 RTase).

Specifically, the cDNA may be labeled with a detectable label. The label may be a material emitting fluorescence, phosphorescence or radiation, although not being limited thereto. Specifically, the label is Cy5 or Cy3. When synthesizing the first strand cDNA, if the synthesis is performed by labeling Cy5 or Cy3 at the 5′-terminal of a primer, a target sequence may be labeled with a detectable fluorescent label. Labeling with a radioactive material may be achieved as follows. When synthesizing the first strand cDNA, if a radioactive isotope such as ³²P, ³⁵S, etc. is added to a reaction solution, the resulting synthesis product may be labeled with the radioactive material.

The step of detecting the degree of hybridization may be performed through capillary electrophoresis, gel electrophoresis, radiation measurement, fluorescence measurement or phosphorescence measurement.

In another exemplary embodiment of the present disclosure, the method for diagnosing cancer may further include a step of diagnosing cancer by comparing the detection result with the standard of the corresponding diagnosed subject and determining the occurrence or growth of cancer in the diagnosed subject.

Meanwhile, the method for diagnosing cancer may be for providing information necessary for diagnosing cancer of the diagnosed subject. By isolating a cell from the diagnosed subject and isolating RNA from the isolated cell of the diagnosed subject in vitro and adding thereto a probe capable of detecting the expression of a base sequence selected from a group consisting of SEQ ID NOS 1-11 or an antibody capable of detecting the expression of an amino acid sequence selected from a group consisting of SEQ ID NOS 12-22, a gene containing a base sequence selected from a group consisting of SEQ ID NOS 1-11 or a protein containing an amino acid sequence selected from a group consisting of SEQ ID NOS 12-22 may be detected.

The method may further include a step of diagnosing the occurrence and growth of cancer in the diagnosed subject by comparing the expression level of the detected gene and protein with the standard of the corresponding diagnosed subject.

The diagnosed subject may be a human, a non-human mammal or an insect.

Although the kit may be used to determine the progression of aging, determine obesity and diagnose cancer separately, as described above, it may also be used to determine the progression of aging, determine the increase in obesity and detect the occurrence of cancer at the same time. The method using the kit for determining the progression of aging, determining the increase in obesity and detecting the occurrence of cancer at the same time may be performed in the same manner as the methods described above using the respective kits.

The aging, obesity and the occurrence or growth of cancer of the diagnosed subject may be determined and diagnosed by comparing the detection result with the standard of the corresponding diagnosed subject.

### MODE FOR INVENTION

Hereinafter, the present disclosure is described in detail through specific examples so that those of ordinary skill in the art to which the present disclosure belongs can easily carry out the present disclosure. However, the present disclosure may be embodied in various different forms and are not limited to the examples.

### Example 1. Preparation of Mutant Drosophila with Expression of T3dh Gene Suppressed

<Preparative process>

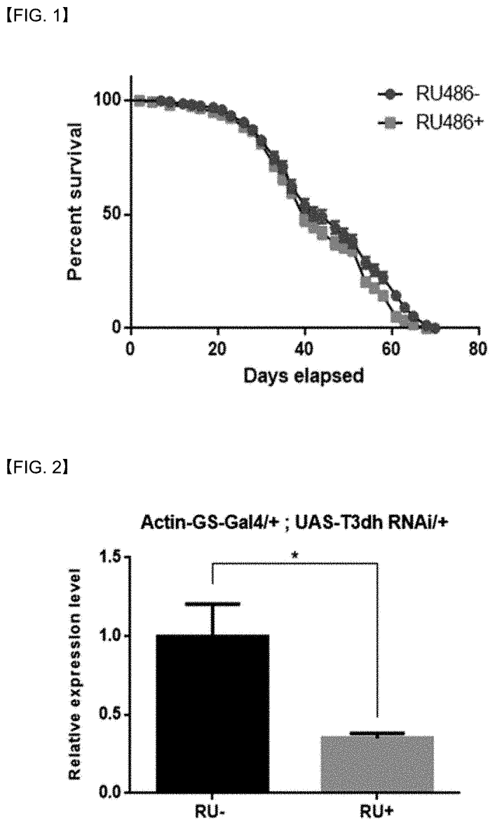

Actin-GS-Gal4 drosophila was mated with wild-type (w1118) drosophila to investigate the effect of RU486 on lifespan. It was confirmed that RU486 has no effect on lifespan (see ‘FIG. 1’).

Actin-GS-Gal4 is expressed through the body of drosophila in the presence of RU486. For UAS-T3dh RNAi, if Gal4 is produced, the expression of T3dh (type III alcohol dehydrogenase, CG3425) is decreased because RNAi interferes with the transcription of T3dh. In order to investigate whether the T3dh expression is decreased in the offspring (F1) drosophila obtained from the mating of Actin-GS-Gal4 and UAS-T3dh RNAi drosophila, the quantity of T3dh mRNA was measured by RT-PCR. As a result, it was found out that the quantity of T3dh mRNA was remarkably decreased when the drosophila was fed with RU486 as compared to when it was not fed with RU486. This results demonstrates that Actin-GS-Gal4 and UAS-T3dh-RNAi operate normally (see ‘FIG. 2’).

### Example 2. Preparation of Mutant Drosophila with Expression of fbp Gene Suppressed

<Preparative process>

Actin-GS-Gal4 drosophila was mated with wild-type (w1118) drosophila to investigate the effect of RU486 on lifespan. It was confirmed that RU486 has no effect on lifespan (see ‘FIG. 3’).

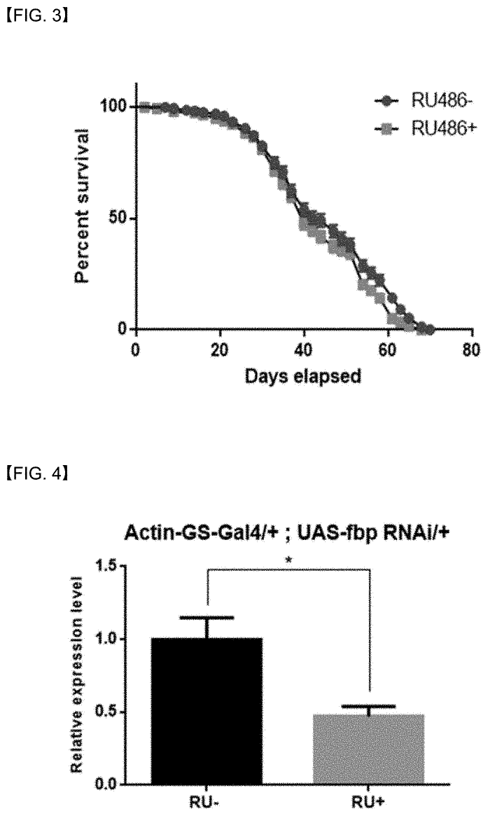

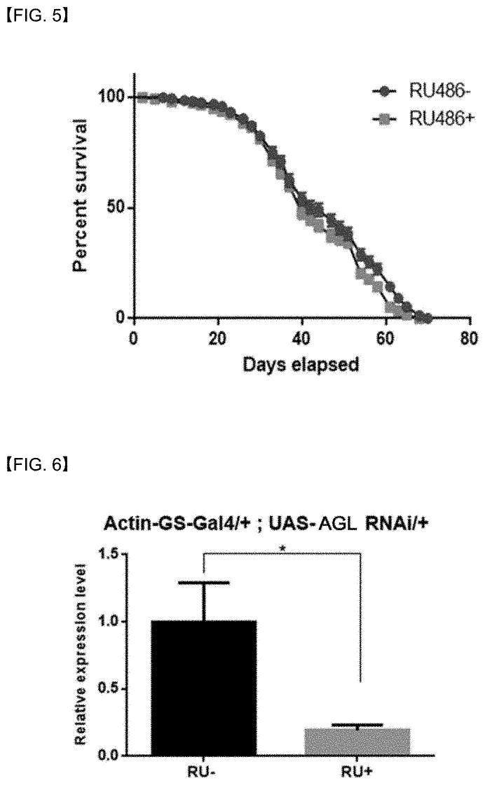

Actin-GS-Gal4 is expressed through the body of drosophila in the presence of RU486. For UAS-fbp RNAi, if Gal4 is produced, the expression of fbp (fructose-1,6-bisphosphatase, CG31692) is decreased because RNAi interferes with the transcription of fbp. In order to investigate whether the fbp expression is decreased in the offspring (F1) drosophila obtained from the mating of Actin-GS-Gal4 and UAS-fbp RNAi drosophila, the quantity of fbp mRNA was measured by RT-PCR. As a result, it was found out that the quantity of fbp m RNA was remarkably decreased when the drosophila was fed with RU486 as compared to when it was not fed with RU486. This results demonstrates that Actin-GS-Gal4 and UAS-fbp RNAi operate normally (see ‘FIG. 4’).

### Example 3. Preparation of Mutant Drosophila with Expression of AGL Gene Suppressed

<Preparative process>

Actin-GS-Gal4 drosophila was mated with wild-type (w1118) drosophila to investigate the effect of RU486 on lifespan. It was confirmed that RU486 has no effect on lifespan (see ‘FIG. 5’).

Actin-GS-Gal4 is expressed through the body of drosophila in the presence of RU486. For UAS-AGL RNAi, if Gal4 is produced, the expression of AGL is decreased because RNAi interferes with the transcription of AGL. In order to investigate whether the AGL expression is decreased in the offspring (F1) drosophila obtained from the mating of Actin-GS-Gal4 and UAS-AGL RNAi drosophila, the quantity of AGL mRNA was measured by RT-PCR. As a result, it was found out that the quantity of AGL mRNA was remarkably decreased when the drosophila was fed with RU486 as compared to when it was not fed with RU486. This results demonstrates that Actin-GS-Gal4 and UAS-AGL RNAi operate normally (see ‘FIG. 6’).

### Relationship of T3dh Gene with Aging, Obesity and Cancer

### Experimental Example 1: RT-PCR of Actin-GS-Gal4>UAS-T3dh RNAi

In order to investigate whether UAS-T3dh RNAi drosophila operates normally, female Actin-GS-Gal4drosophila was mated with male UAS-T3dh RNAi drosophila. Males of the F1 generation were collected and bred with a medium (sucrose 2.5%, glucose 5%, agar 0.7%, corn powder 6.1%, yeast 2.6%, 10% Tegosept 1.6%, water 80.7%) containing RU486 (150 μM) or a medium not containing RU486 for 10 days. Then, reverse transcriptase-mediated polymerase chain reaction (RT-PCR) was conducted to measure the quantity of T3dh mRNA. As a result, it was confirmed that the quantity of T3dh mRNA was statistically significantly decreased in the drosophila bred with the medium containing RU486.

### Experimental Example 2: Measurement of Lifespan of Actin-GS-Gal4>UAS-T3dh RNAi Drosophila Fed with RU486

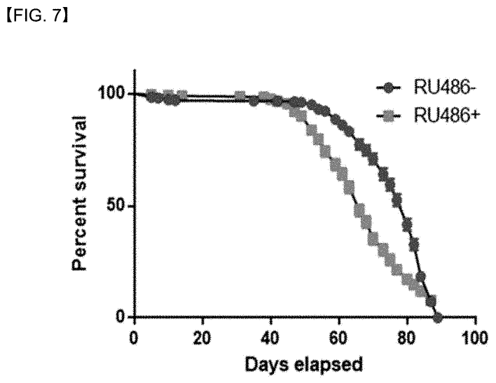

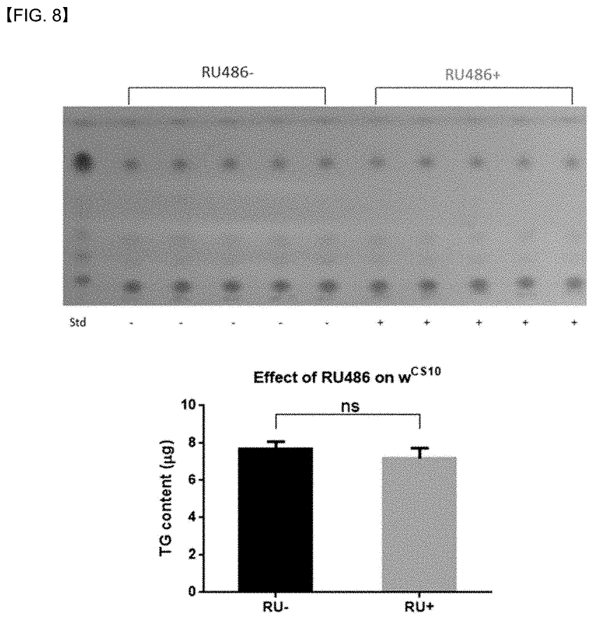

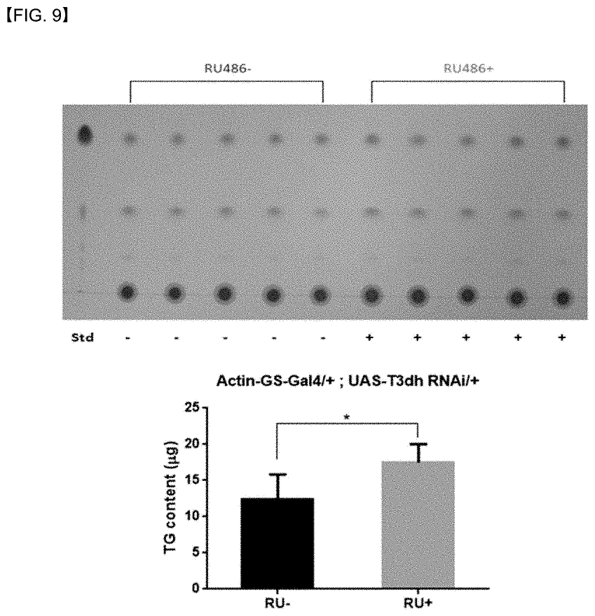

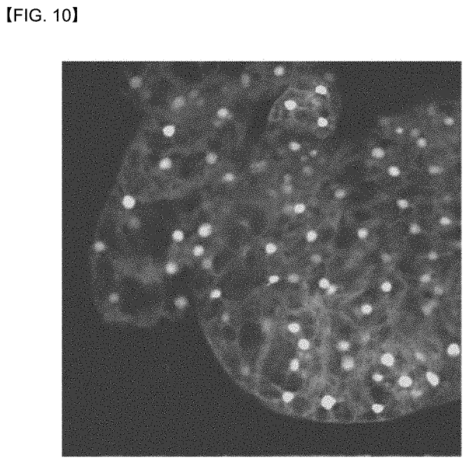

After mating female Actin-GS-Gal4 drosophila with male UAS-T3dh RNAi drosophila, 120 males of the F1 generation were collected and bred with a medium containing RU486 (150 μM) or a medium not containing RU486. Lifespan was measured while breeding them under the condition of 25° C., relative humidity 50% and 12:12 hr light-dark cycle until 6 flies survived. The lifespan measurement was repeated 3 times. As seen from FIG. 7, it was confirmed that the lifespan is decreased, or aging progresses further, as the T3dh expression is decreased (see ‘FIG. 7’).

### Experimental Example 3: Measurement of Triglyceride Content of Actin-GS-Gal4; UAS-T3dh RNAi Drosophila Fed with RU486

After mating female Actin-GS-Gal4 drosophila with male UAS-T3dh RNAi drosophila, about 100 or more males of the F1 generation were collected and bred with a medium containing RU486 (150 μM) or a medium not containing RU486. After 10 days, triglyceride content was measured by thin-layer chromatography (TLC) (‘FIG. 8’ shows the change in triglyceride content of wild-type drosophila fed with RU486). 10 flies per each test group were added to an Eppendorf tube. Centrifugation was conducted after adding 100 μL of an extraction solvent (10 mM Tris, 1 mM EDTA, 0.1% TritonX-100), and crushing for 5 minutes. Then, 5 μL of the obtained supernatant was dropped on a TLC plate. The composition of a TLC eluent for separation of triglyceride was hexane: diethyl ether: acetic acid (70:30:1) and sesame oil (Sigma-Aldrich S3547-250 ML) was used as triglyceride standard. The TLC was dried, stained by putting in an iodine-saturated box and then imaged. The triglyceride band was quantified using the Image J software. As a result, it was confirmed that the triglyceride content was significantly increased in the drosophila fed with RU486, or the T3dh expression-suppressed group (see ‘FIG. 9’).

### Experimental Example 4: Confocal Microscopic Analysis of Fat Body of Actin-GS-Gal4>UAS-T3dh RNAi Drosophila Fed with RU486

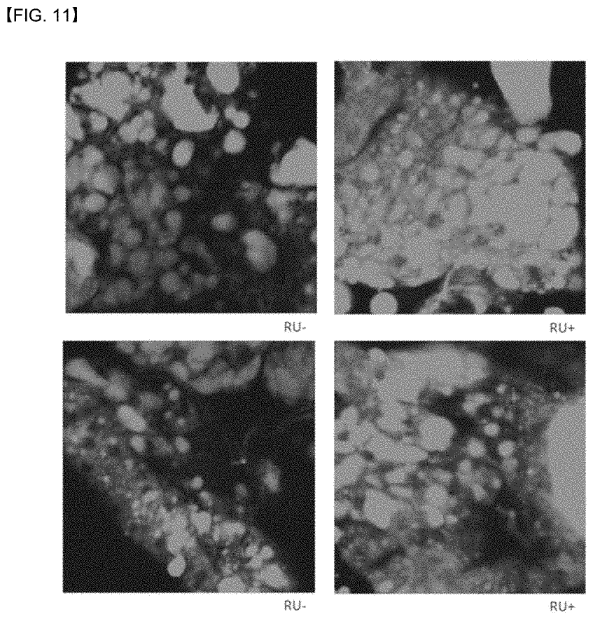

In order to observe the size of lipid droplets, female Actin-GS-Gal4 drosophila was mated with male UAS-T3dh RNAi drosophila and males of the F1 generation were collected and bred with a medium containing RU486 (150 μM) or a medium not containing RU486 for 10 days and then stained with Nile red. The drosophila was dissected and the fat body tissue was fixed in a 4% paraformaldehyde solution in PBS for 30 minutes at room temperature. The solution was washed with PBS, diluted with a 0.5 mg/mL Nile red (Sigma) solution to 1:2,500, stained at room temperature for 30 minutes and then washed twice with distilled water. The stained sample was placed on a slide glass and observed under a confocal microscope after adding an 80% glycerol solution (‘FIG. 10’ shows confocal microscopic images of fat body tissue of Actin-GS-Gal4/UAS-nls.GFP drosophila fed with RU486). As a result, it was confirmed that the size and density of lipid droplets were increased (see ‘FIG. 11’).

### Experimental Example 5: Preparation of Tumor Growth Model Drosophila (UAS-PI3K; c765-Gal4)

Tumor growth model drosophila was prepared by mating c765-Gal4 drosophila with UAS-PI3K drosophila.

### Experimental Example 6: Phenotype Analysis of Tumor Growth Model Drosophila (UAS-PI3K; c765-Gal4)

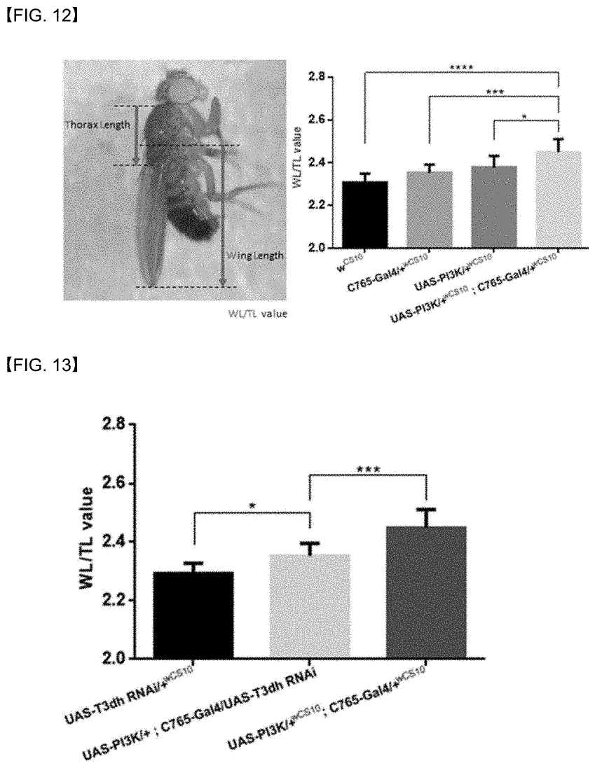

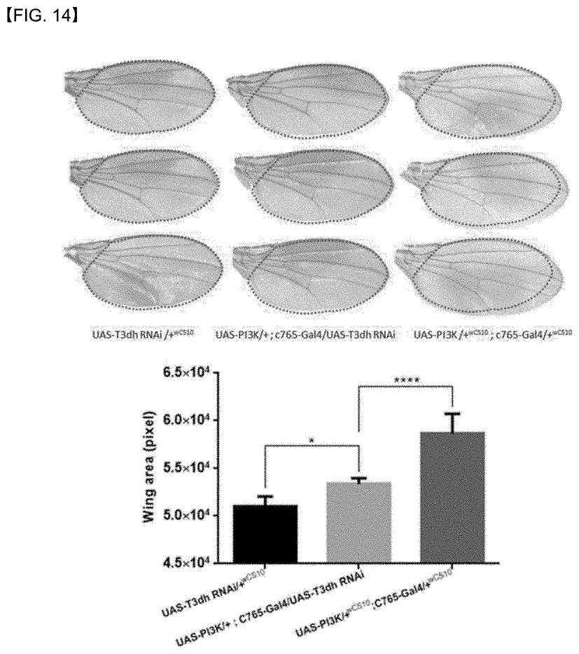

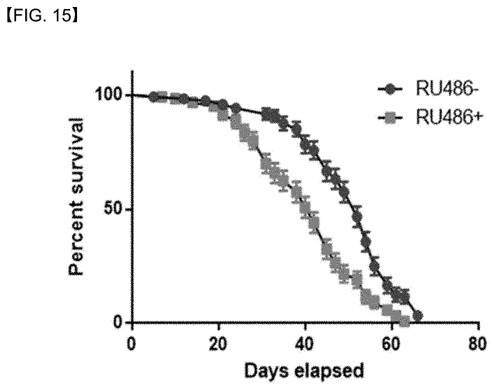

For phenotype analysis of the prepared UAS-PI3K; c765-Gal4 tumor proliferation model drosophila, wing length was compared for wild-type drosophila (CS10) and c765-Gal4/+CS10, UASPI3K/+CS10 and UAS-PI3K/+CS10; c765-Gal4/+CS10 obtained by mating c765-Gal4 with CS10. It was found out that the wing length of UASPI3K/+CS10; c765-Gal4/+CS10 was increased as compared to the three control groups. The wing length with respect to the chest length was measured to compensate for differences among individual flies. It was confirmed that the drosophila model can be sufficiently used as a tumor proliferation model (see ‘FIG. 12’).

### Experimental Example 7: Effect of Suppressed T3dh Gene Expression in Tumor Growth Model Drosophila (UAS-PI3K; c765-Gal4) on Phenotype

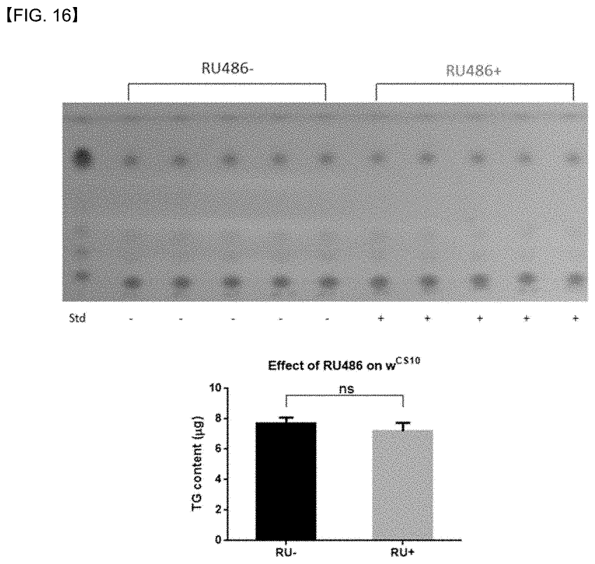

In order to estimate the effect of suppressed T3dh gene expression on tumor growth, the wing phenotype of the F1 generation obtained by mating tumor growth model drosophila (UAS-PI3K; c765-Gal4) with UAS-T3dh RNAi drosophila was investigated. As a result, it was confirmed that the wing length and area increased due to the overexpression of PI3K was significantly decreased as T3dh expression was suppressed (see ‘FIG. 13’ and ‘FIG. 14’).

### Relationship of fbp Gene with Aging, Obesity and Cancer

### Experimental Example 8: RT-PCR of Actin-GS-Gal4>UAS-fbp RNAi

In order to investigate whether UAS-fbp RNAi drosophila operates normally, female Actin-GS-Gal4drosophila was mated with male UAS-fbp RNAi drosophila. Males of the F1 generation were collected and bred with a medium (sucrose 2.5%, glucose 5%, agar 0.7%, corn powder 6.1%, yeast 2.6%, 10% Tegosept 1.6%, water 80.7%) containing RU486 (150 μM) or a medium not containing RU486 for 10 days. Then, reverse transcriptase-mediated polymerase chain reaction (RT-PCR) was conducted to measure the quantity of T3dh mRNA. As a result, it was confirmed that the quantity of fbp mRNA was statistically significantly decreased in the drosophila bred with the medium containing RU486.

### Experimental Example 9: Measurement of Lifespan of Actin-GS-Gal4>UAS-fbp RNAi Drosophila Fed with RU486

After mating female Actin-GS-Gal4 drosophila with male UAS-fbp RNAi drosophila, 120 males of the F1 generation were collected and bred with a medium containing RU486 (150 μM) or a medium not containing RU486. Lifespan was measured while breeding them under the condition of 25° C., relative humidity 50% and 12:12 hr light-dark cycle until 6 flies survived. The lifespan measurement was repeated 3 times. As seen from FIG. 15, it was confirmed that the lifespan is decreased, or aging progresses further, as the fbp expression is decreased (see ‘FIG. 15’).

### Experimental Example 10: Measurement of Triglyceride Content of Actin-GS-Gal4; UAS-fbp RNAi Drosophila Fed with RU486

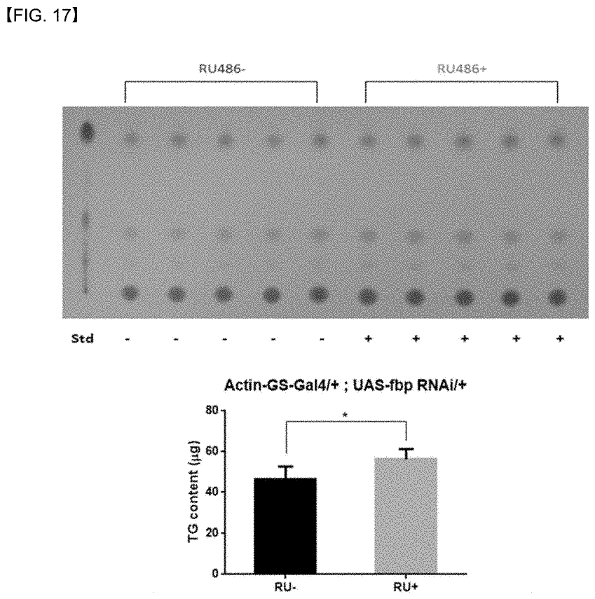

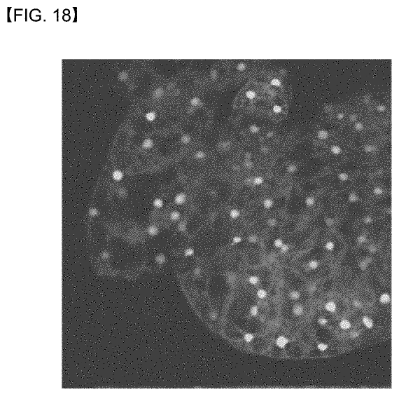

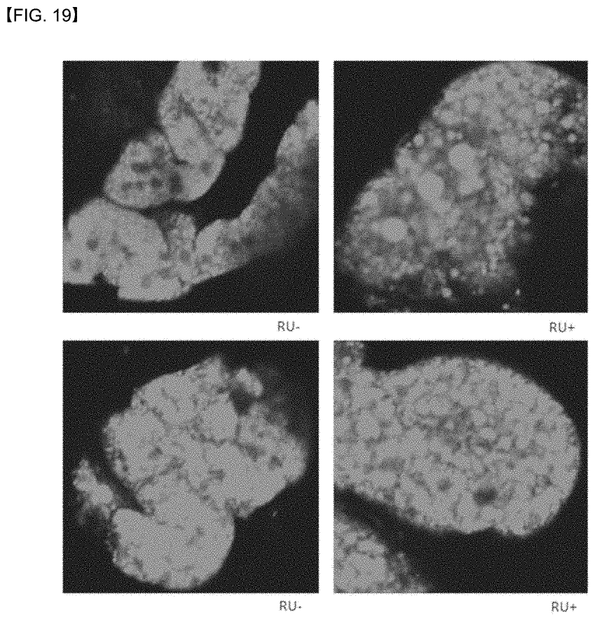

After mating female Actin-GS-Gal4 drosophila with male UAS-fbp RNAi drosophila, about 100 or more males of the F1 generation were collected and bred with a medium containing RU486 (150 μM) or a medium not containing RU486. After 10 days, triglyceride content was measured by thin-layer chromatography (TLC) (‘FIG. 16’ shows the change in triglyceride content of wild-type drosophila fed with RU486). 10 flies per each test group were added to an Eppendorf tube. Centrifugation was conducted after adding 100 μL of an extraction solvent (10 mM Tris, 1 mM EDTA, 0.1% TritonX-100), and crushing for 5 minutes. Then, 5 μL of the obtained supernatant was dropped on a TLC plate. The composition of a TLC eluent for separation of triglyceride was hexane: diethyl ether: acetic acid (70:30:1) and sesame oil (Sigma-Aldrich S3547-250 ML) was used as triglyceride standard. The TLC was dried, stained by putting in an iodine-saturated box and then imaged. The triglyceride band was quantified using the Image J software. As a result, it was confirmed that the triglyceride content was significantly increased in the drosophila fed with RU486, or the fbp expression-suppressed group (see ‘FIG. 17’).

### Experimental Example 11: Confocal Microscopic Analysis of Fat Body of Actin-GS-Gal4>UAS-fbp RNAi Drosophila Fed with RU486

In order to observe the size of lipid droplets, female Actin-GS-Gal4 drosophila was mated with male UAS-fbp RNAi drosophila and males of the F1 generation were collected and bred with a medium containing RU486 (150 μM) or a medium not containing RU486 for 10 days and then stained with Nile red. The drosophila was dissected and the fat body tissue was fixed in a 4% paraformaldehyde solution in PBS for 30 minutes at room temperature. The solution was washed with PBS, diluted with a 0.5 mg/mL Nile red (Sigma) solution to 1:2,500, stained at room temperature for 30 minutes and then washed twice with distilled water. The stained sample was placed on a slide glass and observed under a confocal microscope after adding an 80% glycerol solution (‘FIG. 18’ shows confocal microscopic images of fat body tissue of Actin-GS-Gal4/UAS-nls.GFP drosophila fed with RU486). As a result, it was confirmed that the size and density of lipid droplets were increased (see ‘FIG. 19’).

### Experimental Example 12: Preparation of Tumor Growth Model Drosophila (UAS-Ras85D; c765-Gal4)

Tumor growth model drosophila was prepared by mating c765-Gal4 drosophila with UAS-Ras85D drosophila.

### Experimental Example 13: Phenotype Analysis of Tumor Proliferation Model Drosophila (UAS-Ras85D; c765-Gal4)

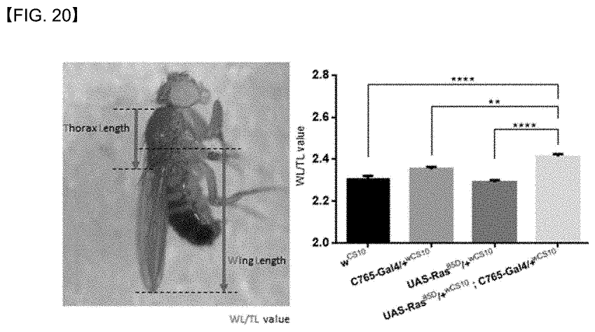

For phenotype analysis of the prepared UAS-Ras85D; c765-Gal4 tumor proliferation model drosophila, wing length was compared for wild-type drosophila (CS10) and c765-Gal4/+CS10; UAS-Ras85D/+CS10 and UAS-Ras85D/+CS10; c765-Gal4/+CS10 obtained by mating c765-Gal4 with CS10. It was found out that the wing length of UAS-Ras85D/+CS10; c765-Gal4/+CS10 was increased as compared to the three control groups. The wing length with respect to the chest length was measured to compensate for differences among individual flies. It was confirmed that the drosophila model can be sufficiently used as a tumor proliferation model (see ‘FIG. 20’).

### Experimental Example 14: Effect of Suppressed fbp Expression in Tumor Proliferation Model Drosophila (UAS-Ras85D; c765-Gal4)

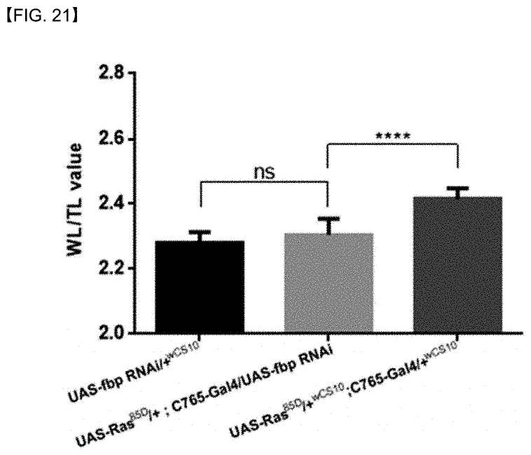

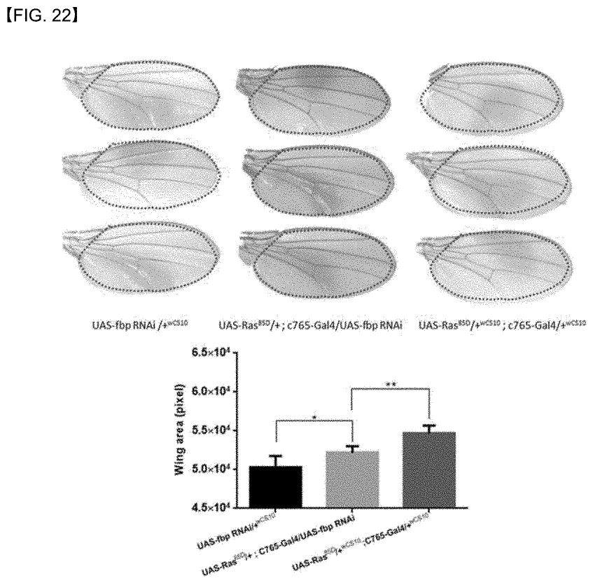

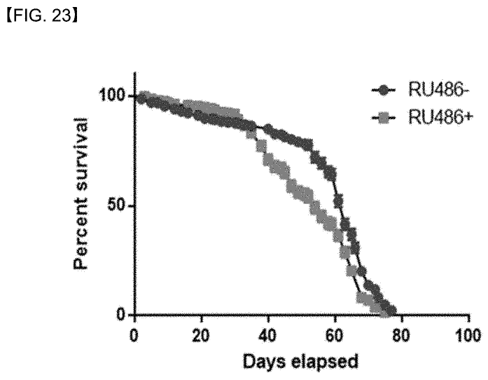

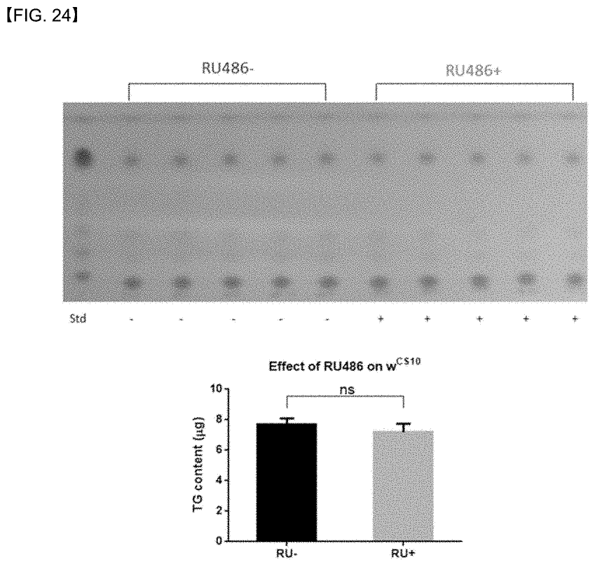

In order to estimate the effect of suppressed fbp gene expression on tumor growth, the wing phenotype of the F1 generation obtained by mating tumor growth model drosophila (UAS-Ras85D; c765-Gal4) with UAS-fbp RNAi drosophila was investigated. As a result, it was confirmed that the wing length and area increased due to the overexpression of Ras85D was significantly decreased as fbp expression was suppressed (see ‘FIG. 21’ and ‘FIG. 22’).

### Relationship of AGL Gene with Aging, Obesity and Cancer

### Experimental Example 15: RT-PCR of Actin-GS-Gal4>UAS-AGL RNAi

In order to investigate whether UAS-AGL RNAi drosophila operates normally, female Actin-GS-Gal4drosophila was mated with male UAS-AGL RNAi drosophila. Males of the F1 generation were collected and bred with a medium (sucrose 2.5%, glucose 5%, agar 0.7%, corn powder 6.1%, yeast 2.6%, 10% Tegosept 1.6%, water 80.7%) containing RU486 (150 μM) or a medium not containing RU486 for 10 days. Then, reverse transcriptase-mediated polymerase chain reaction (RT-PCR) was conducted to measure the quantity of AGL (amylo-alpha-1,6-glucosidase, 4-alphaglucanotransferase, CG9485) mRNA. As a result, it was confirmed that the quantity of AGL mRNA was statistically significantly decreased in the drosophila bred with the medium containing RU486.

### Experimental Example 16: Measurement of Lifespan of Actin-GS-Gal4>UAS-AGL RNAi Drosophila Fed with RU486

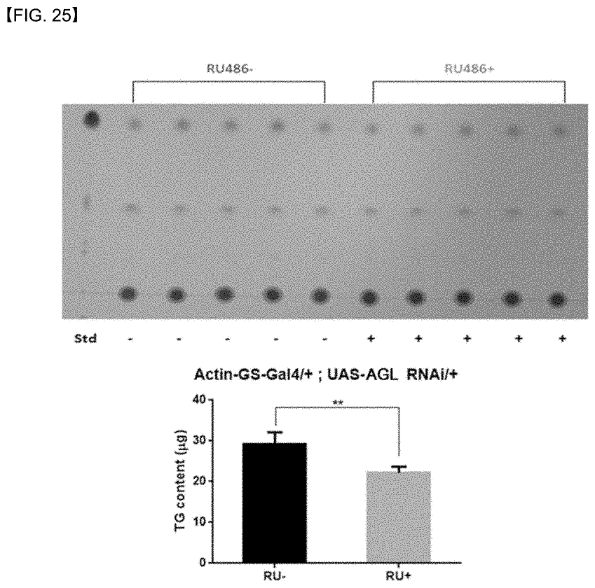

After mating female Actin-GS-Gal4 drosophila with male UAS-AGL RNAi drosophila, 120 males of the F1 generation were collected and bred with a medium containing RU486 (150 μM) or a medium not containing RU486. Lifespan was measured while breeding them under the condition of 25° C., relative humidity 50% and 12:12 hr light-dark cycle until 6 flies survived. The lifespan measurement was repeated 3 times. As seen from FIG. 23, it was confirmed that the lifespan is decreased, or aging progresses further, as the fbp expression is decreased (see ‘FIG. 23’).

### Experimental Example 17: Measurement of Triglyceride Content of Actin-GS-Gal4; UAS-AGL RNAi Drosophila Fed with RU486

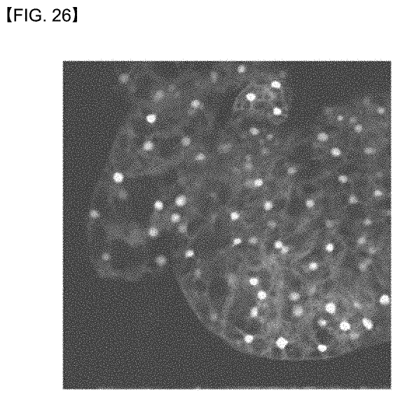

After mating female Actin-GS-Gal4 drosophila with male UAS-AGL RNAi drosophila, about 100 or more males of the F1 generation were collected and bred with a medium containing RU486 (150 μM) or a medium not containing RU486. After 10 days, triglyceride content was measured by thin-layer chromatography (TLC) (‘FIG. 24’ shows the change in triglyceride content of wild-type drosophila fed with RU486). 10 flies per each test group were added to an Eppendorf tube. Centrifugation was conducted after adding 100 μL of an extraction solvent (10 mM Tris, 1 mM EDTA, 0.1% TritonX-100), and crushing for 5 minutes. Then, 5 μL of the obtained supernatant was dropped on a TLC plate. The composition of a TLC eluent for separation of triglyceride was hexane: diethyl ether: acetic acid (70:30:1) and sesame oil (Sigma-Aldrich S3547-250 ML) was used as triglyceride standard. The TLC was dried, stained by putting in an iodine-saturated box and then imaged. The triglyceride band was quantified using the Image J software. As a result, it was confirmed that the triglyceride content was significantly increased in the drosophila fed with RU486, or the AGL expression-suppressed group (see ‘FIG. 25’).

### Experimental Example 18: Confocal Microscopic Analysis of Fat Body of Actin-GS-Gal4>UAS-AGL RNAi Drosophila Fed with RU486

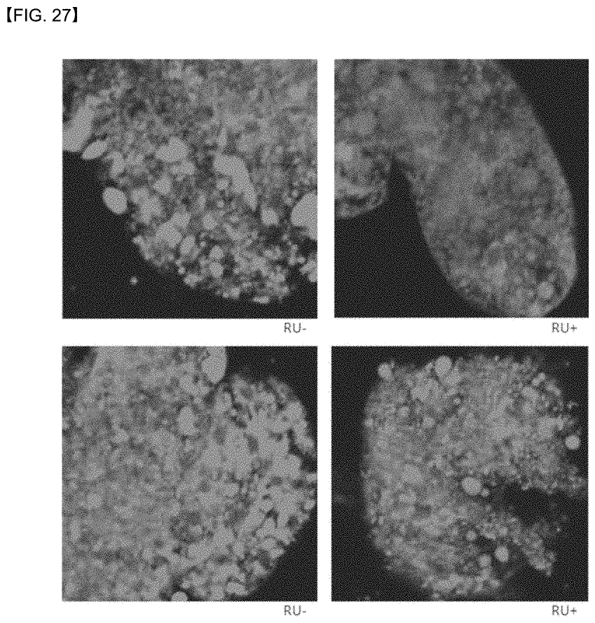

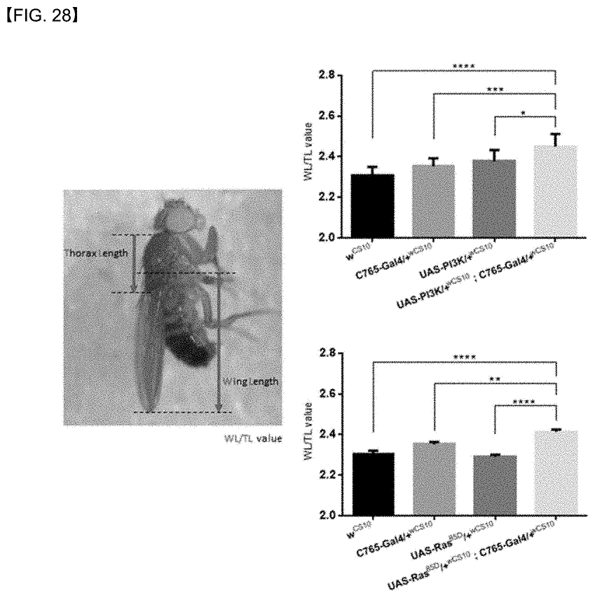

In order to observe the size of lipid droplets, female Actin-GS-Gal4 drosophila was mated with male UAS-AGL RNAi drosophila and males of the F1 generation were collected and bred with a medium containing RU486 (150 μM) or a medium not containing RU486 for 10 days and then stained with Nile red. The drosophila was dissected and the fat body tissue was fixed in a 4% paraformaldehyde solution in PBS for 30 minutes at room temperature. The solution was washed with PBS, diluted with a 0.5 mg/mL Nile red (Sigma) solution to 1:2,500, stained at room temperature for 30 minutes and then washed twice with distilled water. The stained sample was placed on a slide glass and observed under a confocal microscope after adding an 80% glycerol solution (‘FIG. 26’ shows confocal microscopic images of fat body tissue of Actin-GS-Gal4UAS-nls.GFP drosophila fed with RU486). As a result, it was confirmed that the size and density of lipid droplets were increased (see ‘FIG. 27’).

### Experimental Example 19: Preparation of Tumor Growth Model Drosophila (UAS-PI3K; c765-Gal4)

Tumor growth model drosophila was prepared by mating c765-Gal4 drosophila with UAS-PI3K drosophila.

### Experimental Example 20: Preparation of Tumor Proliferation Model Drosophila (UAS-Ras85D; c765-Gal4)

Tumor growth model drosophila was prepared by mating c765-Gal4 drosophila with UAS-Ras85D drosophila.

### Experimental Example 21: Phenotype Analysis of Tumor Proliferation Model Drosophila (UAS-PI3K; c765-Gal4)

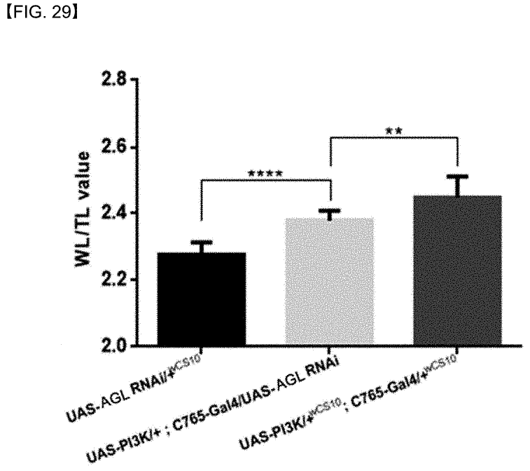

For phenotype analysis of the prepared UAS-PI3K; c765-Gal4 tumor proliferation model drosophila, wing length was compared for wild-type drosophila (CS10) and c765-Gal4/+CS10, UASPI3K/+CS10 and UAS-PI3K/+CS10; c765-Gal4/+CS10 obtained by mating c765-Gal4 with CS10. It was found out that the wing length of UAS-PI3K/+CS10; c765-Gal4/+CS10 was increased as compared to the three control groups. The wing length with respect to the chest length was measured to compensate for differences among individual flies. It was confirmed that the drosophila model can be sufficiently used as a tumor proliferation model (see ‘FIG. 28’).

### Experimental Example 22: Phenotype Analysis of Tumor Proliferation Model Drosophila (UAS-Ras85D; c765-Gal4)

For phenotype analysis of the prepared UAS-Ras85D; c765-Gal4 tumor proliferation model drosophila, wing length was compared for wild-type drosophila (CS10) and c765-Gal4/+CS10; UAS-Ras85D/+CS10 and UAS-Ras85D/+CS10; c765-Gal4/+CS10 obtained by mating c765-Gal4 with CS10. It was found out that the wing length of UAS-Ras85D/+CS10; c765-Gal4/+CS10 was increased as compared to the three control groups. The wing length with respect to the chest length was measured to compensate for differences among individual flies. It was confirmed that the drosophila model can be sufficiently used as a tumor proliferation model (see ‘FIG. 28’).

### Experimental Example 23: Effect of Suppressed AGL Expression in Tumor Proliferation Model Drosophila (UAS-PI3K; c765-Gal4)

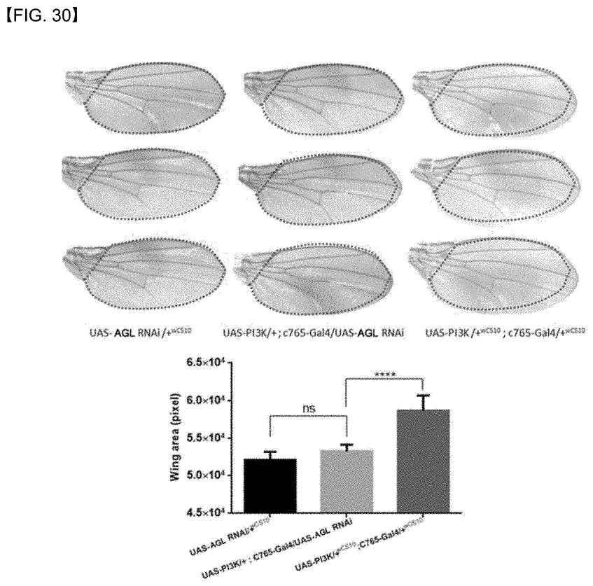

In order to estimate the effect of suppressed AGL gene expression on tumor growth, the wing phenotype of the F1 generation obtained by mating tumor growth model drosophila (UAS-PI3K; c765-Gal4) with UAS-AGL RNAi drosophila was investigated. As a result, it was confirmed that the wing length and area increased due to the overexpression of PI3K was significantly decreased as AGL expression was suppressed (see ‘FIG. 29’ and ‘FIG. 30’).

### Experimental Example 24: Effect of Suppressed AGL Expression in Tumor Proliferation Model Drosophila (UAS-Ras85D; c765-Gal4)

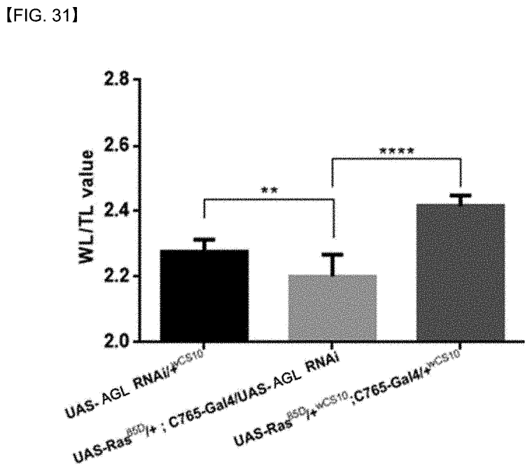

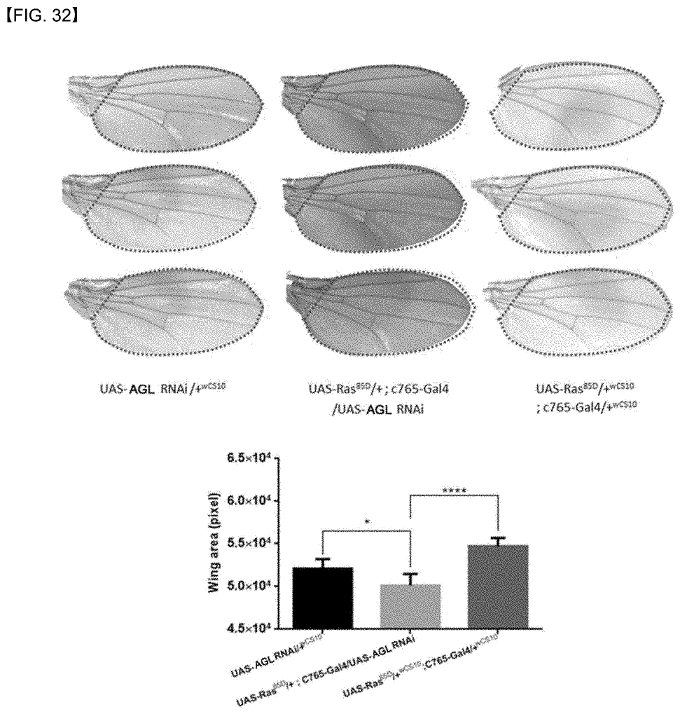

In order to estimate the effect of suppressed AGL gene expression on tumor growth, the wing phenotype of the F1 generation obtained by mating tumor growth model drosophila (UAS-Ras85D; c765-Gal4) with UAS-AGL RNAi drosophila was investigated. As a result, it was confirmed that the wing length and area increased due to the overexpression of Ras85D was significantly decreased as AGL expression was suppressed (see ‘FIG. 31’ and ‘FIG. 32’).

While the specific exemplary embodiments of the present disclosure were described in detail above, it to be understood that the scope of the present disclosure is not limited by them but there may be various modifications within the scope of the present disclosure and they are also included in the scope of the appended claims.

### INDUSTRIAL APPLICABILITY

Because a biomarker of the present disclosure can determine the progression of aging, occurrence of cancer and obesity of a human, a non-human mammal or an insect rapidly and accurately, it provides an important index for new drug development and personalized medicine for various species and can reduce time and cost in the development of biomedicine.

1. A method for determining the progression of aging, comprising:
I) a step of isolating and extracting RNA from a diagnosed subject;
II) a step of hybridizing the isolated RNA or cDNA synthesized therefrom with a biomarker by contacting the RNA or cDNA with a base sequence of SEQ ID NO 1, complementary thereto or mRNAs thereof; and
III) a step of detecting the degree of hybridization between the biomarker and the RNA or cDNA.

2. A method for determining the progression of aging, determining obesity and diagnosing cancer at the same time, comprising:
I) a step of isolating and extracting RNA from a diagnosed subject;
II) a step of hybridizing the isolated RNA or cDNA synthesized therefrom with a biomarker by contacting the RNA or cDNA with a base sequence of SEQ ID NO 1, complementary thereto or mRNAs thereof; and
III) a step of detecting the degree of hybridization between the biomarker and the RNA or cDNA.


SEQUENCE LISTING (22 sequences)

SEQ ID NO: 1 - DNA, 1395 bp, Drosophila sp.
atgtcgcggaagaatgtcttaggtttgatcaacacgatcgtggccaactcctgcaagtgccccgcccattcgcataactatggctcagcagctccaaccgcctcccaaacgggtcgcatggaatacgccttcgagatgtcagcctcgactgttcgatttggaccgggagtaagtgccgaagtgggagccgatcttcgcaacttgggagctcggaaggtttgtttggtcacggataaaaatgtcgtgcaattgccctccgtgaaagtggccttggattcactggctcgtaatggcatcaactacgaagtctatgatgaaacccgggtggagcccacggatgggagcatgtggcatgccgtggagtttgcgcgcggcaaggagttcgatgcatttctggccatcggaggaggatctgccatggatacagccaaggcagctaatctttttagcagtgatgcaaatgcagagtttttggattatgtaaactgcccaattggcagaggcaaggagatatccgtaaaactgaagcccctgattgcgatgcccaccacttcgggaacaggttccgaaaccactggagtagctatatttgattacaagaagttgcatgccaaaactgggatttccagcaagttcctcaaacctactttggctgtgattgatcctctgcacaccctatcccagccgcaacgcgtgatggctttcgctggctttgatgtgttctgccatgccctggagagtttcacggcggtggattacagggaacgcggtttggcgcccagtgatcccagcttgagacccacctaccagggcaggaacccagtgtcggatgtgtgggcacgattcgcattggagacgataaggaaaaactttgtgaacgccatttaccagcccgataacctagaagcccgatcccagatgcacctggcttccacaatggctggcgtgggatttggtaatgccggagtgcacctgtgtcatggcctttcctatcctatttctggaaatgtgagggattacaagccaaagggctactccgcggatcacgctcttattccacacggcctatccgtggtgatcagtgctccggcggtctttgagttcactgctccagcttgtccggatcggcatttggaggctgcccagttgctgggcgcagaagtgcgaggtgtcgaaaaagcagacgccggtcgtcttttggcggacactgtacgcggttttatgcaacgcgcgggcatagaaaatggcctccgagagctgggattctccagcagcgatatacccgccttggtggagggaaccctgccccaggaaaggatcactaagctagcacccagggcgcagacgcaggaaaacctgtcgcaactctttgagaagtccatggaggtctattaa

SEQ ID NO: 2 - DNA, 1005 bp, Drosophila sp.
atgacccaacagaggccagctttcgactccaatgcgatgacgctgacgcgtttcgtgctgcaggagcagcgaaagttcaagagcgccactggcgatctctcccagctgctcaactccatccagaccgccatcaaggctacatcatccgcggtgcggaaggcaggtatcgccaagctccatggattcgctggcgacgtgaatgtccaaggcgaggaggtcaagaaactggacgtgctctccaacgagctgttcatcaacatgctgaagtcatcctataccacatgtctaatggtttccgaggagaacgagaatgtgatcgaggtggaagtggagaaacagggcaaatacatcgtgtgcttcgatcccttggatggatcctccaacatagactgcctggtgtcgatcggttcaatcttcgccatttaccgcaagaaaagcgatggtccgcccacagtggaggatgcactgcagcccggaaatcagctggtggccgccggctacgcgctatacggttcggccacagcaattgtcctgggtctgggttcgggagtgaatggcttcacttatgacccggccatcggagagttcgtgctgaccgatcccaacatgcgggtgccggagaagggaaagatatactctatcaacgagggatatgcagcggattgggaggatggtgtcttcaactacattgcggccaagaaggatcccgccaagggaaagccctatggagcgcggtacgtgggttccatggtcgcggatgtgcatcgcaccattaaatacggcggcatctttatctatccggcaacaaagtccgctcccagcggaaaacttcgtctgctgtacgagtgcgtgcccatggcctatctgatgatccaggctggaggtctggccagcgacggaaagatcagcattttggacattgtgcccaagaagatccacgagcgcagtcccatattcctaggatccaagtccgacgtggaggaggcacttagctacttaaagtga

SEQ ID NO: 3 - DNA, 969 bp, Drosophila sp.
atgacgctgacgcgtttcgtgctgcaggagcagcgaaagttcaagagcgccactggcgatctctcccagctgctcaactccatccagaccgccatcaaggctacatcatccgcggtgcggaaggcaggtatcgccaagctccatggattcgctggcgacgtgaatgtccaaggcgaggaggtcaagaaactggacgtgctctccaacgagctgttcatcaacatgctgaagtcatcctataccacatgtctaatggtttccgaggagaacgagaatgtgatcgaggtggaagtggagaaacagggcaaatacatcgtgtgcttcgatcccttggatggatcctccaacatagactgcctggtgtcgatcggttcaatcttcgccatttaccgcaagaaaagcgatggtccgcccacagtggaggatgcactgcagcccggaaatcagctggtggccgccggctacgcgctatacggttcggccacagcaattgtcctgggtctgggttcgggagtgaatggcttcacttatgacccggccatcggagagttcgtgctgaccgatcccaacatgcgggtgccggagaagggaaagatatactctatcaacgagggatatgcagcggattgggaggatggtgtcttcaactacattgcggccaagaaggatcccgccaagggaaagccctatggagcgcggtacgtgggttccatggtcgcggatgtgcatcgcaccattaaatacggcggcatctttatctatccggcaacaaagtccgctcccagcggaaaacttcgtctgctgtacgagtgcgtgcccatggcctatctgatgatccaggctggaggtctggccagcgacggaaagatcagcattttggacattgtgcccaagaagatccacgagcgcagtcccatattcctaggatccaagtccgacgtggaggaggcacttagctacttaaagtga

SEQ ID NO: 4 - DNA, 969 bp, Drosophila sp.; identical to SEQ ID NO: 3

SEQ ID NO: 5 - DNA, 1032 bp, Drosophila sp.
atggcggccagtagcggtgattccaaaatgacccaacagaggccagctttcgactccaatgcgatgacgctgacgcgtttcgtgctgcaggagcagcgaaagttcaagagcgccactggcgatctctcccagctgctcaactccatccagaccgccatcaaggctacatcatccgcggtgcggaaggcaggtatcgccaagctccatggattcgctggcgacgtgaatgtccaaggcgaggaggtcaagaaactggacgtgctctccaacgagctgttcatcaacatgctgaagtcatcctataccacatgtctaatggtttccgaggagaacgagaatgtgatcgaggtggaagtggagaaacagggcaaatacatcgtgtgcttcgatcccttggatggatcctccaacatagactgcctggtgtcgatcggttcaatcttcgccatttaccgcaagaaaagcgatggtccgcccacagtggaggatgcactgcagcccggaaatcagctggtggccgccggctacgcgctatacggttcggccacagcaattgtcctgggtctgggttcgggagtgaatggcttcacttatgacccggccatcggagagttcgtgctgaccgatcccaacatgcgggtgccggagaagggaaagatatactctatcaacgagggatatgcagcggattgggaggatggtgtcttcaactacattgcggccaagaaggatcccgccaagggaaagccctatggagcgcggtacgtgggttccatggtcgcggatgtgcatcgcaccattaaatacggcggcatctttatctatccggcaacaaagtccgctcccagcggaaaacttcgtctgctgtacgagtgcgtgcccatggcctatctgatgatccaggctggaggtctggccagcgacggaaagatcagcattttggacattgtgcccaagaagatccacgagcgcagtcccatattcctaggatccaagtccgacgtggaggaggcacttagctacttaaagtga

SEQ ID NO: 6 - DNA, 4890 bp, Drosophila sp.
atgcgttatcagctcttccgatggctttatggactaatagcgactgttgataacgaaccgctaccgttacaaatcaaaagcgaagaggaagcgttcggggaaaacaagaagaagaagcagctggcgagtctggagaatgccatctcccccggaaaattggattcagtcgcgattagaaccgttccaagtgcaaatgcgaatgcaatgggcaatgcaggcagtgccgagatcgtatcaaatcaaatcatccgccgccgtgagatgagcaccatgggcaaggagacggagtcgcatagcatacccatcagcgagggccaggatgcggagcatatcctgtaccgtctgaagcggggttccaagctgagtgttcatccggatgcctcgttgctgggcaggaagatcgtattgtacaccaactatcccgccgagggacagaagtttgtgcgtacggagtatcgcgtgctgggatggcaactcagcaatggcaagcagattacctctgtaatgcatccggaggcccatgtggtagatactgacattcgtagtcaggtggagctcaacatgtccggcacctaccacttttacttccggtatctggaaagacccgacactggctgctccggagcagatggagctctatatgtgcaagtggagcccacgctgcatgttgggccgcctggcgcccagaagaccattcccttggactcggtgcgctgccaaacggtgctggccaagctcctgggaccactggacacttgggagcctaagctgcgcgtggcaaaggaggccggatacaatgtgatccacttcactcccatccaggagttgggtggctcccgatcctgctattcgctgcgtgatcaactcaaggtcaactcccattttgcaccccaaaagggaggtaagatcagttttgaggatgtggagaaggtcatcaagaagtgccgccaggagtggggggtggcctccatctgcgacatcgtgctcaatcacaccgccaacgagtccgattggctactacagcatccggatgccacctactcatgcgccacctgtccctacctccgccccgccttcctgctggacgccacattcgcccagtgcggagcggacatagccgagggcagcctggagcatgtaggcgtacctgcggtcatcgagcaggagtgccatttggaagcgttaaagtaccagttgcacacctcctacatgtccaaggtcaatatacacgagctgtatcagtgcgatgtgatgaagtacgtgaacgagttcatgtcccaggtgcgaactcgcgagccaccgaagaatgtggccaacgagtgtcgcttccaggaaatccaactgatacaggacccgcaatatcgacgcttggccagcaccattaatttcgagctggcgctggagatctttaatgctttccatggcgactgcttcgatgaggagtcacggttccgcaagtgcgccgaaaccctccgccggcatctcgatgccctcaacgatcgtgtgcgctgcgaggttcaaggctacataaactatgcgatagacaatgttttggccggagttcgctacgaaagagtccagggcgatgggcccagagtgaaggagatctccgagaagcactcggtctttatggtctactttacgcataccggcacccagggaaaatcgctcactgagatcgaggcggatatgtataccaaggctggagagttcttcatggcccacaacggttgggtcatgggatacagcgatcctctgagggattttgctgaggagcaacctggacgcgccaatgtctacctcaagcgggagctcatctcgtggggcgatagtgtgaagttgcgcttcggcagacggccggaggacagtccctacctatggcagcacatgaccgagtacgttcagaccacagcgcgcatcttcgacggtgtgcgattggacaactgccactccacgccattgcacgtagctgagtaccttctcgatgcagctcgtaagatcaacccagagctgtatgtggtggctgaactgttcaccaattccgattacaccgacaatgtgtttgtgaaccgattgggtatcacctccttgatccgtgaagctctttccgcttgggactcccacgagcaaggccgcttagtgtatcggtatggaggagtgcctgtaggtggtttccaagcaaactcatcgcgccacgaggccaccagtgtcgctcatgccctcttcctggacctcacccacgataatccgtctccggtggagaagcgttccgtgtacgatcttctaccatcggcggcactggtttccatggcatgctgtgccacaggaagtaaccgtggttacgacgaactggtcccccatcatatccatgtcgtagatgaggaacgcacctaccaagaatggggcaaaggtgttgactcgaagtccggaattatgggtgccaaaagagcactgaatttgctgcatggacagctcgcagaggagggatttagccaagtttacgtagaccagatggatcccaacgtagttgcagttactcgtcattcgccaatcacgcatcagtcagttattctcgtggcccacactgcctttggctatccctctcctaatgccggacccaccggaatccgcccgctgcgtttcgagggtgtgctggacgagatcatcctggaggccagcttgaccatgcagagtgacaagccattcgatcgtcctgctccattcaaaaaggatccgaatgtaatcaacggctttactcagttccagttgaatctgcaggagcacatcccgctggctaagtcgacagtgtttcagacccaagcctattcggatggcaacaacacggagctaaactttgccaatctacgacccggcactgtggtggccatcagagtgtctatgcatcctggtcctcgcaccagtttcgataagctccagaaaatctcggctgccctgcgtattggatctggcgaggagtattcccagctgcaggctatcgtctccaagttggatctggtggcacttagtggtgcccttttcagctgcgatgatgaggagagggatcttggcaaaggtggcaccgcctacgacattcccaactttggaaagatcgtttactgcggattgcaaggattcatttccctgttgacggagatttcgcctaaaaatgacttgggacatccgctttgtaacaatctgcgcgacggaaactggatgatggattacatttctgatcgcctaactagttatgaagacctgaaaccactctccgcctggttcaaagctacctttgagccactgaagaatattccacgctacctcataccctgctactttgatgccattgtcagcggggtttacaatgtgctcatcaaccaggtcaacgaactaatgccagacttcattaagaatggccacagtttcccacaatccctggccctgtccacgttgcagttcctctccgtttgcaagtcggccaatctgccgggattcagtcctgctctaagtccacccaagcctccaaagcaatgtgtgactctatctgctggtctgccgcatttctcaacgggctatatgcggtgctggggccgtgataccttcatcgctctgcgtggctccatgttcctcactggccgttacaacgaggctcgcttcatcatcattggatttggtcagacccttcgacacggactcattccgaatcttttggacagcggcagcaagccgagattcaactgccgcgatgctatctggtggtggatgtactgcatcaagcagtatgtggaggatgcccccaagggtgccgagatcctgaaggacaaggtgtcccgcatatttccgtacgacgatgccgatgcccatgctccaggtgccttcgaccaacttctcttcgacgtgatgcaggaggcactgcaggtgcattttcagggcttgcagtatagggagcgcaatgcaggctatgagatcgatgcgcacatggtggaccagggctttaacaatcagatcggaattcacccggagactggctttgtattcggaggcaataacttcaactgcggcacttggatggacaaaatgggatcctcacagaaggccggaaacaagggacgtccaagtacgccgcgcgacggatcagccgtggagctcgttggcctccagtatgccgtactccggtttatgcaaagcctagccgaaaaggaggttatcccgtacaccggcgtggaacgaaagggtccatcgggcgaagtgaccaagtggagctacaaggagtgggcggatcgcatcaagaacaactttgacaagtatttctttgtatccgaatcggaaacctgctcggtggccaacaagaagcttatctacaaggacagctatggagccacccagagttggacggactaccagctgcgatgcaacttccccatcaccttgaccgtggctcccgacctgtgcaatcctcagaatgcctggcgtgcactggagcgcgccaaaaagtatcttctgggaccgctgggcatgaagacgatggatcccgaggactggaactatagggccaactatgacaactcaaatgactccaccgattgcactgtagcccatggcgcaaactaccaccaggggccggagtgggtatggcccatcggtttttacctgcgggcgcgcctgatcttcgccaaaaagtgtggccatttggacgagaccattgccgagacctgggccatactacgggcccatctccgagagctacagacatcccattggcgcggattgcccgagttgaccaacgataatggctcctactgcggtgactcctgtcgcacgcaggcctggagtgttgctgccattttggaagtcctgtacgatctgcactccttgggagcagacgtggcctaa

SEQ ID NO: 7 - DNA, 4629 bp, Drosophila sp.
atgagcaccatgggcaaggagacggagtcgcatagcatacccatcagcgagggccaggatgcggagcatatcctgtaccgtctgaagcggggttccaagctgagtgttcatccggatgcctcgttgctgggcaggaagatcgtattgtacaccaactatcccgccgagggacagaagtttgtgcgtacggagtatcgcgtgctgggatggcaactcagcaatggcaagcagattacctctgtaatgcatccggaggcccatgtggtagatactgacattcgtagtcaggtggagctcaacatgtccggcacctaccacttttacttccggtatctggaaagacccgacactggctgctccggagcagatggagctctatatgtgcaagtggagcccacgctgcatgttgggccgcctggcgcccagaagaccattcccttggactcggtgcgctgccaaacggtgctggccaagctcctgggaccactggacacttgggagcctaagctgcgcgtggcaaaggaggccggatacaatgtgatccacttcactcccatccaggagttgggtggctcccgatcctgctattcgctgcgtgatcaactcaaggtcaactcccattttgcaccccaaaagggaggtaagatcagttttgaggatgtggagaaggtcatcaagaagtgccgccaggagtggggggtggcctccatctgcgacatcgtgctcaatcacaccgccaacgagtccgattggctactacagcatccggatgccacctactcatgcgccacctgtccctacctccgccccgccttcctgctggacgccacattcgcccagtgcggagcggacatagccgagggcagcctggagcatgtaggcgtacctgcggtcatcgagcaggagtgccatttggaagcgttaaagtaccagttgcacacctcctacatgtccaaggtcaatatacacgagctgtatcagtgcgatgtgatgaagtacgtgaacgagttcatgtcccaggtgcgaactcgcgagccaccgaagaatgtggccaacgagtgtcgcttccaggaaatccaactgatacaggacccgcaatatcgacgcttggccagcaccattaatttcgagctggcgctggagatctttaatgctttccatggcgactgcttcgatgaggagtcacggttccgcaagtgcgccgaaaccctccgccggcatctcgatgccctcaacgatcgtgtgcgctgcgaggttcaaggctacataaactatgcgatagacaatgttttggccggagttcgctacgaaagagtccagggcgatgggcccagagtgaaggagatctccgagaagcactcggtctttatggtctactttacgcataccggcacccagggaaaatcgctcactgagatcgaggcggatatgtataccaaggctggagagttcttcatggcccacaacggttgggtcatgggatacagcgatcctctgagggattttgctgaggagcaacctggacgcgccaatgtctacctcaagcgggagctcatctcgtggggcgatagtgtgaagttgcgcttcggcagacggccggaggacagtccctacctatggcagcacatgaccgagtacgttcagaccacagcgcgcatcttcgacggtgtgcgattggacaactgccactccacgccattgcacgtagctgagtaccttctcgatgcagctcgtaagatcaacccagagctgtatgtggtggctgaactgttcaccaattccgattacaccgacaatgtgtttgtgaaccgattgggtatcacctccttgatccgtgaagctctttccgcttgggactcccacgagcaaggccgcttagtgtatcggtatggaggagtgcctgtaggtggtttccaagcaaactcatcgcgccacgaggccaccagtgtcgctcatgccctcttcctggacctcacccacgataatccgtctccggtggagaagcgttccgtgtacgatcttctaccatcggcggcactggtttccatggcatgctgtgccacaggaagtaaccgtggttacgacgaactggtcccccatcatatccatgtcgtagatgaggaacgcacctaccaagaatggggcaaaggtgttgactcgaagtccggaattatgggtgccaaaagagcactgaatttgctgcatggacagctcgcagaggagggatttagccaagtttacgtagaccagatggatcccaacgtagttgcagttactcgtcattcgccaatcacgcatcagtcagttattctcgtggcccacactgcctttggctatccctctcctaatgccggacccaccggaatccgcccgctgcgtttcgagggtgtgctggacgagatcatcctggaggccagcttgaccatgcagagtgacaagccattcgatcgtcctgctccattcaaaaaggatccgaatgtaatcaacggctttactcagttccagttgaatctgcaggagcacatcccgctggctaagtcgacagtgtttcagacccaagcctattcggatggcaacaacacggagctaaactttgccaatctacgacccggcactgtggtggccatcagagtgtctatgcatcctggtcctcgcaccagtttcgataagctccagaaaatctcggctgccctgcgtattggatctggcgaggagtattcccagctgcaggctatcgtctccaagttggatctggtggcacttagtggtgcccttttcagctgcgatgatgaggagagggatcttggcaaaggtggcaccgcctacgacattcccaactttggaaagatcgtttactgcggattgcaaggattcatttccctgttgacggagatttcgcctaaaaatgacttgggacatccgctttgtaacaatctgcgcgacggaaactggatgatggattacatttctgatcgcctaactagttatgaagacctgaaaccactctccgcctggttcaaagctacctttgagccactgaagaatattccacgctacctcataccctgctactttgatgccattgtcagcggggtttacaatgtgctcatcaaccaggtcaacgaactaatgccagacttcattaagaatggccacagtttcccacaatccctggccctgtccacgttgcagttcctctccgtttgcaagtcggccaatctgccgggattcagtcctgctctaagtccacccaagcctccaaagcaatgtgtgactctatctgctggtctgccgcatttctcaacgggctatatgcggtgctggggccgtgataccttcatcgctctgcgtggctccatgttcctcactggccgttacaacgaggctcgcttcatcatcattggatttggtcagacccttcgacacggactcattccgaatcttttggacagcggcagcaagccgagattcaactgccgcgatgctatctggtggtggatgtactgcatcaagcagtatgtggaggatgcccccaagggtgccgagatcctgaaggacaaggtgtcccgcatatttccgtacgacgatgccgatgcccatgctccaggtgccttcgaccaacttctcttcgacgtgatgcaggaggcactgcaggtgcattttcagggcttgcagtatagggagcgcaatgcaggctatgagatcgatgcgcacatggtggaccagggctttaacaatcagatcggaattcacccggagactggctttgtattcggaggcaataacttcaactgcggcacttggatggacaaaatgggatcctcacagaaggccggaaacaagggacgtccaagtacgccgcgcgacggatcagccgtggagctcgttggcctccagtatgccgtactccggtttatgcaaagcctagccgaaaaggaggttatcccgtacaccggcgtggaacgaaagggtccatcgggcgaagtgaccaagtggagctacaaggagtgggcggatcgcatcaagaacaactttgacaagtatttctttgtatccgaatcggaaacctgctcggtggccaacaagaagcttatctacaaggacagctatggagccacccagagttggacggactaccagctgcgatgcaacttccccatcaccttgaccgtggctcccgacctgtgcaatcctcagaatgcctggcgtgcactggagcgcgccaaaaagtatcttctgggaccgctgggcatgaagacgatggatcccgaggactggaactatagggccaactatgacaactcaaatgactccaccgattgcactgtagcccatggcgcaaactaccaccaggggccggagtgggtatggcccatcggtttttacctgcgggcgcgcctgatcttcgccaaaaagtgtggccatttggacgagaccattgccgagacctgggccatactacgggcccatctccgagagctacagacatcccattggcgcggattgcccgagttgaccaacgataatggctcctactgcggtgactcctgtcgcacgcaggcctggagtgttgctgccattttggaagtcctgtacgatctgcactccttgggagcagacgtggcctaa

SEQ ID NO: 8 - DNA, 4641 bp, Drosophila sp.
atgcctttggctatgagcaccatgggcaaggagacggagtcgcatagcatacccatcagcgagggccaggatgcggagcatatcctgtaccgtctgaagcggggttccaagctgagtgttcatccggatgcctcgttgctgggcaggaagatcgtattgtacaccaactatcccgccgagggacagaagtttgtgcgtacggagtatcgcgtgctgggatggcaactcagcaatggcaagcagattacctctgtaatgcatccggaggcccatgtggtagatactgacattcgtagtcaggtggagctcaacatgtccggcacctaccacttttacttccggtatctggaaagacccgacactggctgctccggagcagatggagctctatatgtgcaagtggagcccacgctgcatgttgggccgcctggcgcccagaagaccattcccttggactcggtgcgctgccaaacggtgctggccaagctcctgggaccactggacacttgggagcctaagctgcgcgtggcaaaggaggccggatacaatgtgatccacttcactcccatccaggagttgggtggctcccgatcctgctattcgctgcgtgatcaactcaaggtcaactcccattttgcaccccaaaagggaggtaagatcagttttgaggatgtggagaaggtcatcaagaagtgccgccaggagtggggggtggcctccatctgcgacatcgtgctcaatcacaccgccaacgagtccgattggctactacagcatccggatgccacctactcatgcgccacctgtccctacctccgccccgccttcctgctggacgccacattcgcccagtgcggagcggacatagccgagggcagcctggagcatgtaggcgtacctgcggtcatcgagcaggagtgccatttggaagcgttaaagtaccagttgcacacctcctacatgtccaaggtcaatatacacgagctgtatcagtgcgatgtgatgaagtacgtgaacgagttcatgtcccaggtgcgaactcgcgagccaccgaagaatgtggccaacgagtgtcgcttccaggaaatccaactgatacaggacccgcaatatcgacgcttggccagcaccattaatttcgagctggcgctggagatctttaatgctttccatggcgactgcttcgatgaggagtcacggttccgcaagtgcgccgaaaccctccgccggcatctcgatgccctcaacgatcgtgtgcgctgcgaggttcaaggctacataaactatgcgatagacaatgttttggccggagttcgctacgaaagagtccagggcgatgggcccagagtgaaggagatctccgagaagcactcggtctttatggtctactttacgcataccggcacccagggaaaatcgctcactgagatcgaggcggatatgtataccaaggctggagagttcttcatggcccacaacggttgggtcatgggatacagcgatcctctgagggattttgctgaggagcaacctggacgcgccaatgtctacctcaagcgggagctcatctcgtggggcgatagtgtgaagttgcgcttcggcagacggccggaggacagtccctacctatggcagcacatgaccgagtacgttcagaccacagcgcgcatcttcgacggtgtgcgattggacaactgccactccacgccattgcacgtagctgagtaccttctcgatgcagctcgtaagatcaacccagagctgtatgtggtggctgaactgttcaccaattccgattacaccgacaatgtgtttgtgaaccgattgggtatcacctccttgatccgtgaagctctttccgcttgggactcccacgagcaaggccgcttagtgtatcggtatggaggagtgcctgtaggtggtttccaagcaaactcatcgcgccacgaggccaccagtgtcgctcatgccctcttcctggacctcacccacgataatccgtctccggtggagaagcgttccgtgtacgatcttctaccatcggcggcactggtttccatggcatgctgtgccacaggaagtaaccgtggttacgacgaactggtcccccatcatatccatgtcgtagatgaggaacgcacctaccaagaatggggcaaaggtgttgactcgaagtccggaattatgggtgccaaaagagcactgaatttgctgcatggacagctcgcagaggagggatttagccaagtttacgtagaccagatggatcccaacgtagttgcagttactcgtcattcgccaatcacgcatcagtcagttattctcgtggcccacactgcctttggctatccctctcctaatgccggacccaccggaatccgcccgctgcgtttcgagggtgtgctggacgagatcatcctggaggccagcttgaccatgcagagtgacaagccattcgatcgtcctgctccattcaaaaaggatccgaatgtaatcaacggctttactcagttccagttgaatctgcaggagcacatcccgctggctaagtcgacagtgtttcagacccaagcctattcggatggcaacaacacggagctaaactttgccaatctacgacccggcactgtggtggccatcagagtgtctatgcatcctggtcctcgcaccagtttcgataagctccagaaaatctcggctgccctgcgtattggatctggcgaggagtattcccagctgcaggctatcgtctccaagttggatctggtggcacttagtggtgcccttttcagctgcgatgatgaggagagggatcttggcaaaggtggcaccgcctacgacattcccaactttggaaagatcgtttactgcggattgcaaggattcatttccctgttgacggagatttcgcctaaaaatgacttgggacatccgctttgtaacaatctgcgcgacggaaactggatgatggattacatttctgatcgcctaactagttatgaagacctgaaaccactctccgcctggttcaaagctacctttgagccactgaagaatattccacgctacctcataccctgctactttgatgccattgtcagcggggtttacaatgtgctcatcaaccaggtcaacgaactaatgccagacttcattaagaatggccacagtttcccacaatccctggccctgtccacgttgcagttcctctccgtttgcaagtcggccaatctgccgggattcagtcctgctctaagtccacccaagcctccaaagcaatgtgtgactctatctgctggtctgccgcatttctcaacgggctatatgcggtgctggggccgtgataccttcatcgctctgcgtggctccatgttcctcactggccgttacaacgaggctcgcttcatcatcattggatttggtcagacccttcgacacggactcattccgaatcttttggacagcggcagcaagccgagattcaactgccgcgatgctatctggtggtggatgtactgcatcaagcagtatgtggaggatgcccccaagggtgccgagatcctgaaggacaaggtgtcccgcatatttccgtacgacgatgccgatgcccatgctccaggtgccttcgaccaacttctcttcgacgtgatgcaggaggcactgcaggtgcattttcagggcttgcagtatagggagcgcaatgcaggctatgagatcgatgcgcacatggtggaccagggctttaacaatcagatcggaattcacccggagactggctttgtattcggaggcaataacttcaactgcggcacttggatggacaaaatgggatcctcacagaaggccggaaacaagggacgtccaagtacgccgcgcgacggatcagccgtggagctcgttggcctccagtatgccgtactccggtttatgcaaagcctagccgaaaaggaggttatcccgtacaccggcgtggaacgaaagggtccatcgggcgaagtgaccaagtggagctacaaggagtgggcggatcgcatcaagaacaactttgacaagtatttctttgtatccgaatcggaaacctgctcggtggccaacaagaagcttatctacaaggacagctatggagccacccagagttggacggactaccagctgcgatgcaacttccccatcaccttgaccgtggctcccgacctgtgcaatcctcagaatgcctggcgtgcactggagcgcgccaaaaagtatcttctgggaccgctgggcatgaagacgatggatcccgaggactggaactatagggccaactatgacaactcaaatgactccaccgattgcactgtagcccatggcgcaaactaccaccaggggccggagtgggtatggcccatcggtttttacctgcgggcgcgcctgatcttcgccaaaaagtgtggccatttggacgagaccattgccgagacctgggccatactacgggcccatctccgagagctacagacatcccattggcgcggattgcccgagttgaccaacgataatggctcctactgcggtgactcctgtcgcacgcaggcctggagtgttgctgccattttggaagtcctgtacgatctgcactccttgggagcagacgtggcctaa

SEQ ID NO: 9 - DNA, 4692 bp, Drosophila sp.
atgagcaccatgggcaaggagacggagtcgcatagcatacccatcagcgagggccaggatgcggagcatatcctgtaccgtctgaagcggggttccaagctgagtgttcatccggatgcctcgttgctgggcaggaagatcgtattgtacaccaactatcccgccgagggacagaagtttgtgcgtacggagtatcgcgtgctgggatggcaactcagcaatggcaagcagattacctctgtaatgcatccggaggcccatgtggtagatactgacattcgtagtcaggtggagctcaacatgtccggcacctaccacttttacttccggtatctggaaaggttcgaaactatacttttccgcattgattttgctttagcattaatgtgtgtcaacattttcagacccgacactggctgctccggagcagatggagctctatatgtgcaagtggagcccacgctgcatgttgggccgcctggcgcccagaagaccattcccttggactcggtgcgctgccaaacggtgctggccaagctcctgggaccactggacacttgggagcctaagctgcgcgtggcaaaggaggccggatacaatgtgatccacttcactcccatccaggagttgggtggctcccgatcctgctattcgctgcgtgatcaactcaaggtcaactcccattttgcaccccaaaagggaggtaagatcagttttgaggatgtggagaaggtcatcaagaagtgccgccaggagtggggggtggcctccatctgcgacatcgtgctcaatcacaccgccaacgagtccgattggctactacagcatccggatgccacctactcatgcgccacctgtccctacctccgccccgccttcctgctggacgccacattcgcccagtgcggagcggacatagccgagggcagcctggagcatgtaggcgtacctgcggtcatcgagcaggagtgccatttggaagcgttaaagtaccagttgcacacctcctacatgtccaaggtcaatatacacgagctgtatcagtgcgatgtgatgaagtacgtgaacgagttcatgtcccaggtgcgaactcgcgagccaccgaagaatgtggccaacgagtgtcgcttccaggaaatccaactgatacaggacccgcaatatcgacgcttggccagcaccattaatttcgagctggcgctggagatctttaatgctttccatggcgactgcttcgatgaggagtcacggttccgcaagtgcgccgaaaccctccgccggcatctcgatgccctcaacgatcgtgtgcgctgcgaggttcaaggctacataaactatgcgatagacaatgttttggccggagttcgctacgaaagagtccagggcgatgggcccagagtgaaggagatctccgagaagcactcggtctttatggtctactttacgcataccggcacccagggaaaatcgctcactgagatcgaggcggatatgtataccaaggctggagagttcttcatggcccacaacggttgggtcatgggatacagcgatcctctgagggattttgctgaggagcaacctggacgcgccaatgtctacctcaagcgggagctcatctcgtggggcgatagtgtgaagttgcgcttcggcagacggccggaggacagtccctacctatggcagcacatgaccgagtacgttcagaccacagcgcgcatcttcgacggtgtgcgattggacaactgccactccacgccattgcacgtagctgagtaccttctcgatgcagctcgtaagatcaacccagagctgtatgtggtggctgaactgttcaccaattccgattacaccgacaatgtgtttgtgaaccgattgggtatcacctccttgatccgtgaagctctttccgcttgggactcccacgagcaaggccgcttagtgtatcggtatggaggagtgcctgtaggtggtttccaagcaaactcatcgcgccacgaggccaccagtgtcgctcatgccctcttcctggacctcacccacgataatccgtctccggtggagaagcgttccgtgtacgatcttctaccatcggcggcactggtttccatggcatgctgtgccacaggaagtaaccgtggttacgacgaactggtcccccatcatatccatgtcgtagatgaggaacgcacctaccaagaatggggcaaaggtgttgactcgaagtccggaattatgggtgccaaaagagcactgaatttgctgcatggacagctcgcagaggagggatttagccaagtttacgtagaccagatggatcccaacgtagttgcagttactcgtcattcgccaatcacgcatcagtcagttattctcgtggcccacactgcctttggctatccctctcctaatgccggacccaccggaatccgcccgctgcgtttcgagggtgtgctggacgagatcatcctggaggccagcttgaccatgcagagtgacaagccattcgatcgtcctgctccattcaaaaaggatccgaatgtaatcaacggctttactcagttccagttgaatctgcaggagcacatcccgctggctaagtcgacagtgtttcagacccaagcctattcggatggcaacaacacggagctaaactttgccaatctacgacccggcactgtggtggccatcagagtgtctatgcatcctggtcctcgcaccagtttcgataagctccagaaaatctcggctgccctgcgtattggatctggcgaggagtattcccagctgcaggctatcgtctccaagttggatctggtggcacttagtggtgcccttttcagctgcgatgatgaggagagggatcttggcaaaggtggcaccgcctacgacattcccaactttggaaagatcgtttactgcggattgcaaggattcatttccctgttgacggagatttcgcctaaaaatgacttgggacatccgctttgtaacaatctgcgcgacggaaactggatgatggattacatttctgatcgcctaactagttatgaagacctgaaaccactctccgcctggttcaaagctacctttgagccactgaagaatattccacgctacctcataccctgctactttgatgccattgtcagcggggtttacaatgtgctcatcaaccaggtcaacgaactaatgccagacttcattaagaatggccacagtttcccacaatccctggccctgtccacgttgcagttcctctccgtttgcaagtcggccaatctgccgggattcagtcctgctctaagtccacccaagcctccaaagcaatgtgtgactctatctgctggtctgccgcatttctcaacgggctatatgcggtgctggggccgtgataccttcatcgctctgcgtggctccatgttcctcactggccgttacaacgaggctcgcttcatcatcattggatttggtcagacccttcgacacggactcattccgaatcttttggacagcggcagcaagccgagattcaactgccgcgatgctatctggtggtggatgtactgcatcaagcagtatgtggaggatgcccccaagggtgccgagatcctgaaggacaaggtgtcccgcatatttccgtacgacgatgccgatgcccatgctccaggtgccttcgaccaacttctcttcgacgtgatgcaggaggcactgcaggtgcattttcagggcttgcagtatagggagcgcaatgcaggctatgagatcgatgcgcacatggtggaccagggctttaacaatcagatcggaattcacccggagactggctttgtattcggaggcaataacttcaactgcggcacttggatggacaaaatgggatcctcacagaaggccggaaacaagggacgtccaagtacgccgcgcgacggatcagccgtggagctcgttggcctccagtatgccgtactccggtttatgcaaagcctagccgaaaaggaggttatcccgtacaccggcgtggaacgaaagggtccatcgggcgaagtgaccaagtggagctacaaggagtgggcggatcgcatcaagaacaactttgacaagtatttctttgtatccgaatcggaaacctgctcggtggccaacaagaagcttatctacaaggacagctatggagccacccagagttggacggactaccagctgcgatgcaacttccccatcaccttgaccgtggctcccgacctgtgcaatcctcagaatgcctggcgtgcactggagcgcgccaaaaagtatcttctgggaccgctgggcatgaagacgatggatcccgaggactggaactatagggccaactatgacaactcaaatgactccaccgattgcactgtagcccatggcgcaaactaccaccaggggccggagtgggtatggcccatcggtttttacctgcgggcgcgcctgatcttcgccaaaaagtgtggccatttggacgagaccattgccgagacctgggccatactacgggcccatctccgagagctacagacatcccattggcgcggattgcccgagttgaccaacgataatggctcctactgcggtgactcctgtcgcacgcaggcctggagtgttgctgccattttggaagtcctgtacgatctgcactccttgggagcagacgtggcctaa

SEQ ID NO: 10 - DNA, 4629 bp, Drosophila sp.; identical to SEQ ID NO: 7

SEQ ID NO: 11 - DNA, 4629 bp, Drosophila sp.; identical to SEQ ID NO: 7

SEQ ID NO: 12 - protein, 464 aa, Drosophila sp.
MSRKNVLGLINTIVANSCKCPAHSHNYGSAAPTASQTGRMEYAFEMSASTVRFGPGVSAEVGADLRNLGARKVCLVTDKNVVQLPSVKVALDSLARNGINYEVYDETRVEPTDGSMWHAVEFARGKEFDAFLAIGGGSAMDTAKAANLFSSDANAEFLDYVNCPIGRGKEISVKLKPLIAMPTTSGTGSETTGVAIFDYKKLHAKTGISSKFLKPTLAVIDPLHTLSQPQRVMAFAGFDVFCHALESFTAVDYRERGLAPSDPSLRPTYQGRNPVSDVWARFALETIRKNFVNAIYQPDNLEARSQMHLASTMAGVGFGNAGVHLCHGLSYPISGNVRDYKPKGYSADHALIPHGLSVVISAPAVFEFTAPACPDRHLEAAQLLGAEVRGVEKADAGRLLADTVRGFMQRAGIENGLRELGFSSSDIPALVEGTLPQERITKLAPRAQTQENLSQLFEKSMEVY

SEQ ID NO: 13 - protein, 334 aa, Drosophila sp.
MTQQRPAFDSNAMTLTRFVLQEQRKFKSATGDLSQLLNSIQTAIKATSSAVRKAGIAKLHGFAGDVNVQGEEVKKLDVLSNELFINMLKSSYTTCLMVSEENENVIEVEVEKQGKYIVCFDPLDGSSNIDCLVSIGSIFAIYRKKSDGPPTVEDALQPGNQLVAAGYALYGSATAIVLGLGSGVNGFTYDPAIGEFVLTDPNMRVPEKGKIYSINEGYAADWEDGVFNYIAAKKDPAKGKPYGARYVGSMVADVHRTIKYGGIFIYPATKSAPSGKLRLLYECVPMAYLMIQAGGLASDGKISILDIVPKKIHERSPIFLGSKSDVEEALSYLK

SEQ ID NO: 14 - protein, 322 aa, Drosophila sp.
MTLTRFVLQEQRKFKSATGDLSQLLNSIQTAIKATSSAVRKAGIAKLHGFAGDVNVQGEEVKKLDVLSNELFINMLKSSYTTCLMVSEENENVIEVEVEKQGKYIVCFDPLDGSSNIDCLVSIGSIFAIYRKKSDGPPTVEDALQPGNQLVAAGYALYGSATAIVLGLGSGVNGFTYDPAIGEFVLTDPNMRVPEKGKIYSINEGYAADWEDGVFNYIAAKKDPAKGKPYGARYVGSMVADVHRTIKYGGIFIYPATKSAPSGKLRLLYECVPMAYLMIQAGGLASDGKISILDIVPKKIHERSPIFLGSKSDVEEALSYLK

SEQ ID NO: 15 - protein, 322 aa, Drosophila sp.; identical to SEQ ID NO: 14

SEQ ID NO: 16 - protein, 343 aa, Drosophila sp.
MAASSGDSKMTQQRPAFDSNAMTLTRFVLQEQRKFKSATGDLSQLLNSIQTAIKATSSAVRKAGIAKLHGFAGDVNVQGEEVKKLDVLSNELFINMLKSSYTTCLMVSEENENVIEVEVEKQGKYIVCFDPLDGSSNIDCLVSIGSIFAIYRKKSDGPPTVEDALQPGNQLVAAGYALYGSATAIVLGLGSGVNGFTYDPAIGEFVLTDPNMRVPEKGKIYSINEGYAADWEDGVFNYIAAKKDPAKGKPYGARYVGSMVADVHRTIKYGGIFIYPATKSAPSGKLRLLYECVPMAYLMIQAGGLASDGKISILDIVPKKIHERSPIFLGSKSDVEEALSYLK

SEQ ID NO: 17 - protein, 1629 aa, Drosophila sp.
MRYQLFRWLYGLIATVDNEPLPLQIKSEEEAFGENKKKKQLASLENAISPGKLDSVAIRTVPSANANAMGNAGSAEIVSNQIIRRREMSTMGKETESHSIPISEGQDAEHILYRLKRGSKLSVHPDASLLGRKIVLYTNYPAEGQKFVRTEYRVLGWQLSNGKQITSVMHPEAHVVDTDIRSQVELNMSGTYHFYFRYLERPDTGCSGADGALYVQVEPTLHVGPPGAQKTIPLDSVRCQTVLAKLLGPLDTWEPKLRVAKEAGYNVIHFTPIQELGGSRSCYSLRDQLKVNSHFAPQKGGKISFEDVEKVIKKCRQEWGVASICDIVLNHTANESDWLLQHPDATYSCATCPYLRPAFLLDATFAQCGADIAEGSLEHVGVPAVIEQECHLEALKYQLHTSYMSKVNIHELYQCDVMKYVNEFMSQVRTREPPKNVANECRFQEIQLIQDPQYRRLASTINFELALEIFNAFHGDCFDEESRFRKCAETLRRHLDALNDRVRCEVQGYINYAIDNVLAGVRYERVQGDGPRVKEISEKHSVFMVYFTHTGTQGKSLTEIEADMYTKAGEFFMAHNGWVMGYSDPLRDFAEEQPGRANVYLKRELISWGDSVKLRFGRRPEDSPYLWQHMTEYVQTTARIFDGVRLDNCHSTPLHVAEYLLDAARKINPELYVVAELFTNSDYTDNVFVNRLGITSLIREALSAWDSHEQGRLVYRYGGVPVGGFQANSSRHEATSVAHALFLDLTHDNPSPVEKRSVYDLLPSAALVSMACCATGSNRGYDELVPHHIHVVDEERTYQEWGKGVDSKSGIMGAKRALNLLHGQLAEEGFSQVYVDQMDPNVVAVTRHSPITHQSVILVAHTAFGYPSPNAGPTGIRPLRFEGVLDEIILEASLTMQSDKPFDRPAPFKKDPNVINGFTQFQLNLQEHIPLAKSTVFQTQAYSDGNNTELNFANLRPGTVVAIRVSMHPGPRTSFDKLQKISAALRIGSGEEYSQLQAIVSKLDLVALSGALFSCDDEERDLGKGGTAYDIPNFGKIVYCGLQGFISLLTEISPKNDLGHPLCNNLRDGNWMMDYISDRLTSYEDLKPLSAWFKATFEPLKNIPRYLIPCYFDAIVSGVYNVLINQVNELMPDFIKNGHSFPQSLALSTLQFLSVCKSANLPGFSPALSPPKPPKQCVTLSAGLPHFSTGYMRCWGRDTFIALRGSMFLTGRYNEARFIIIGFGQTLRHGLIPNLLDSGSKPRFNCRDAIWWWMYCIKQYVEDAPKGAEILKDKVSRIFPYDDADAHAPGAFDQLLFDVMQEALQVHFQGLQYRERNAGYEIDAHMVDQGFNNQIGIHPETGFVFGGNNFNCGTWMDKMGSSQKAGNKGRPSTPRDGSAVELVGLQYAVLRFMQSLAEKEVIPYTGVERKGPSGEVTKWSYKEWADRIKNNFDKYFFVSESETCSVANKKLIYKDSYGATQSWTDYQLRCNFPITLTVAPDLCNPQNAWRALERAKKYLLGPLGMKTMDPEDWNYRANYDNSNDSTDCTVAHGANYHQGPEWVWPIGFYLRARLIFAKKCGHLDETIAETWAILRAHLRELQTSHWRGLPELTNDNGSYCGDSCRTQAWSVAAILEVLYDLHSLGADVA

SEQ ID NO: 18 - protein, 1542 aa, Drosophila sp.
MSTMGKETESHSIPISEGQDAEHILYRLKRGSKLSVHPDASLLGRKIVLYTNYPAEGQKFVRTEYRVLGWQLSNGKQITSVMHPEAHVVDTDIRSQVELNMSGTYHFYFRYLERPDTGCSGADGALYVQVEPTLHVGPPGAQKTIPLDSVRCQTVLAKLLGPLDTWEPKLRVAKEAGYNVIHFTPIQELGGSRSCYSLRDQLKVNSHFAPQKGGKISFEDVEKVIKKCRQEWGVASICDIVLNHTANESDWLLQHPDATYSCATCPYLRPAFLLDATFAQCGADIAEGSLEHVGVPAVIEQECHLEALKYQLHTSYMSKVNIHELYQCDVMKYVNEFMSQVRTREPPKNVANECRFQEIQLIQDPQYRRLASTINFELALEIFNAFHGDCFDEESRFRKCAETLRRHLDALNDRVRCEVQGYINYAIDNVLAGVRYERVQGDGPRVKEISEKHSVFMVYFTHTGTQGKSLTEIEADMYTKAGEFFMAHNGWVMGYSDPLRDFAEEQPGRANVYLKRELISWGDSVKLRFGRRPEDSPYLWQHMTEYVQTTARIFDGVRLDNCHSTPLHVAEYLLDAARKINPELYVVAELFTNSDYTDNVFVNRLGITSLIREALSAWDSHEQGRLVYRYGGVPVGGFQANSSRHEATSVAHALFLDLTHDNPSPVEKRSVYDLLPSAALVSMACCATGSNRGYDELVPHHIHVVDEERTYQEWGKGVDSKSGIMGAKRALNLLHGQLAEEGFSQVYVDQMDPNVVAVTRHSPITHQSVILVAHTAFGYPSPNAGPTGIRPLRFEGVLDEIILEASLTMQSDKPFDRPAPFKKDPNVINGFTQFQLNLQEHIPLAKSTVFQTQAYSDGNNTELNFANLRPGTVVAIRVSMHPGPRTSFDKLQKISAALRIGSGEEYSQLQAIVSKLDLVALSGALFSCDDEERDLGKGGTAYDIPNFGKIVYCGLQGFISLLTEISPKNDLGHPLCNNLRDGNWMMDYISDRLTSYEDLKPLSAWFKATFEPLKNIPRYLIPCYFDAIVSGVYNVLINQVNELMPDFIKNGHSFPQSLALSTLQFLSVCKSANLPGFSPALSPPKPPKQCVTLSAGLPHFSTGYMRCWGRDTFIALRGSMFLTGRYNEARFIIIGFGQTLRHGLIPNLLDSGSKPRFNCRDAIWWWMYCIKQYVEDAPKGAEILKDKVSRIFPYDDADAHAPGAFDQLLFDVMQEALQVHFQGLQYRERNAGYEIDAHMVDQGFNNQIGIHPETGFVFGGNNFNCGTWMDKMGSSQKAGNKGRPSTPRDGSAVELVGLQYAVLRFMQSLAEKEVIPYTGVERKGPSGEVTKWSYKEWADRIKNNFDKYFFVSESETCSVANKKLIYKDSYGATQSWTDYQLRCNFPITLTVAPDLCNPQNAWRALERAKKYLLGPLGMKTMDPEDWNYRANYDNSNDSTDCTVAHGANYHQGPEWVWPIGFYLRARLIFAKKCGHLDETIAETWAILRAHLRELQTSHWRGLPELTNDNGSYCGDSCRTQAWSVAAILEVLYDLHSLGADVA

SEQ ID NO: 19 - protein, 1546 aa, Drosophila sp.
MPLAMSTMGKETESHSIPISEGQDAEHILYRLKRGSKLSVHPDASLLGRKIVLYTNYPAEGQKFVRTEYRVLGWQLSNGKQITSVMHPEAHVVDTDIRSQVELNMSGTYHFYFRYLERPDTGCSGADGALYVQVEPTLHVGPPGAQKTIPLDSVRCQTVLAKLLGPLDTWEPKLRVAKEAGYNVIHFTPIQELGGSRSCYSLRDQLKVNSHFAPQKGGKISFEDVEKVIKKCRQEWGVASICDIVLNHTANESDWLLQHPDATYSCATCPYLRPAFLLDATFAQCGADIAEGSLEHVGVPAVIEQECHLEALKYQLHTSYMSKVNIHELYQCDVMKYVNEFMSQVRTREPPKNVANECRFQEIQLIQDPQYRRLASTINFELALEIFNAFHGDCFDEESRFRKCAETLRRHLDALNDRVRCEVQGYINYAIDNVLAGVRYERVQGDGPRVKEISEKHSVFMVYFTHTGTQGKSLTEIEADMYTKAGEFFMAHNGWVMGYSDPLRDFAEEQPGRANVYLKRELISWGDSVKLRFGRRPEDSPYLWQHMTEYVQTTARIFDGVRLDNCHSTPLHVAEYLLDAARKINPELYVVAELFTNSDYTDNVFVNRLGITSLIREALSAWDSHEQGRLVYRYGGVPVGGFQANSSRHEATSVAHALFLDLTHDNPSPVEKRSVYDLLPSAALVSMACCATGSNRGYDELVPHHIHVVDEERTYQEWGKGVDSKSGIMGAKRALNLLHGQLAEEGFSQVYVDQMDPNVVAVTRHSPITHQSVILVAHTAFGYPSPNAGPTGIRPLRFEGVLDEIILEASLTMQSDKPFDRPAPFKKDPNVINGFTQFQLNLQEHIPLAKSTVFQTQAYSDGNNTELNFANLRPGTVVAIRVSMHPGPRTSFDKLQKISAALRIGSGEEYSQLQAIVSKLDLVALSGALFSCDDEERDLGKGGTAYDIPNFGKIVYCGLQGFISLLTEISPKNDLGHPLCNNLRDGNWMMDYISDRLTSYEDLKPLSAWFKATFEPLKNIPRYLIPCYFDAIVSGVYNVLINQVNELMPDFIKNGHSFPQSLALSTLQFLSVCKSANLPGFSPALSPPKPPKQCVTLSAGLPHFSTGYMRCWGRDTFIALRGSMFLTGRYNEARFIIIGFGQTLRHGLIPNLLDSGSKPRFNCRDAIWWWMYCIKQYVEDAPKGAEILKDKVSRIFPYDDADAHAPGAFDQLLFDVMQEALQVHFQGLQYRERNAGYEIDAHMVDQGFNNQIGIHPETGFVFGGNNFNCGTWMDKMGSSQKAGNKGRPSTPRDGSAVELVGLQYAVLRFMQSLAEKEVIPYTGVERKGPSGEVTKWSYKEWADRIKNNFDKYFFVSESETCSVANKKLIYKDSYGATQSWTDYQLRCNFPITLTVAPDLCNPQNAWRALERAKKYLLGPLGMKTMDPEDWNYRANYDNSNDSTDCTVAHGANYHQGPEWVWPIGFYLRARLIFAKKCGHLDETIAETWAILRAHLRELQTSHWRGLPELTNDNGSYCGDSCRTQAWSVAAILEVLYDLHSLGADVA

SEQ ID NO: 20 - protein, 1563 aa, Drosophila sp.
MSTMGKETESHSIPISEGQDAEHILYRLKRGSKLSVHPDASLLGRKIVLYTNYPAEGQKFVRTEYRVLGWQLSNGKQITSVMHPEAHVVDTDIRSQVELNMSGTYHFYFRYLERFETILFRIDFALALMCVNIFRPDTGCSGADGALYVQVEPTLHVGPPGAQKTIPLDSVRCQTVLAKLLGPLDTWEPKLRVAKEAGYNVIHFTPIQELGGSRSCYSLRDQLKVNSHFAPQKGGKISFEDVEKVIKKCRQEWGVASICDIVLNHTANESDWLLQHPDATYSCATCPYLRPAFLLDATFAQCGADIAEGSLEHVGVPAVIEQECHLEALKYQLHTSYMSKVNIHELYQCDVMKYVNEFMSQVRTREPPKNVANECRFQEIQLIQDPQYRRLASTINFELALEIFNAFHGDCFDEESRFRKCAETLRRHLDALNDRVRCEVQGYINYAIDNVLAGVRYERVQGDGPRVKEISEKHSVFMVYFTHTGTQGKSLTEIEADMYTKAGEFFMAHNGWVMGYSDPLRDFAEEQPGRANVYLKRELISWGDSVKLRFGRRPEDSPYLWQHMTEYVQTTARIFDGVRLDNCHSTPLHVAEYLLDAARKINPELYVVAELFTNSDYTDNVFVNRLGITSLIREALSAWDSHEQGRLVYRYGGVPVGGFQANSSRHEATSVAHALFLDLTHDNPSPVEKRSVYDLLPSAALVSMACCATGSNRGYDELVPHHIHVVDEERTYQEWGKGVDSKSGIMGAKRALNLLHGQLAEEGFSQVYVDQMDPNVVAVTRHSPITHQSVILVAHTAFGYPSPNAGPTGIRPLRFEGVLDEIILEASLTMQSDKPFDRPAPFKKDPNVINGFTQFQLNLQEHIPLAKSTVFQTQAYSDGNNTELNFANLRPGTVVAIRVSMHPGPRTSFDKLQKISAALRIGSGEEYSQLQAIVSKLDLVALSGALFSCDDEERDLGKGGTAYDIPNFGKIVYCGLQGFISLLTEISPKNDLGHPLCNNLRDGNWMMDYISDRLTSYEDLKPLSAWFKATFEPLKNIPRYLIPCYFDAIVSGVYNVLINQVNELMPDFIKNGHSFPQSLALSTLQFLSVCKSANLPGFSPALSPPKPPKQCVTLSAGLPHFSTGYMRCWGRDTFIALRGSMFLTGRYNEARFIIIGFGQTLRHGLIPNLLDSGSKPRFNCRDAIWWWMYCIKQYVEDAPKGAEILKDKVSRIFPYDDADAHAPGAFDQLLFDVMQEALQVHFQGLQYRERNAGYEIDAHMVDQGFNNQIGIHPETGFVFGGNNFNCGTWMDKMGSSQKAGNKGRPSTPRDGSAVELVGLQYAVLRFMQSLAEKEVIPYTGVERKGPSGEVTKWSYKEWADRIKNNFDKYFFVSESETCSVANKKLIYKDSYGATQSWTDYQLRCNFPITLTVAPDLCNPQNAWRALERAKKYLLGPLGMKTMDPEDWNYRANYDNSNDSTDCTVAHGANYHQGPEWVWPIGFYLRARLIFAKKCGHLDETIAETWAILRAHLRELQTSHWRGLPELTNDNGSYCGDSCRTQAWSVAAILEVLYDLHSLGADVA

SEQ ID NO: 21 - protein, 1542 aa, Drosophila sp.; identical to SEQ ID NO: 18

SEQ ID NO: 22 - protein, 1542 aa, Drosophila sp.; identical to SEQ ID NO: 18